(19) United States
(12) Patent Application Publication
(10) Pub. No.: US 2008/0193448 A1
(43) Pub. Date: Aug. 14, 2008

(54) Combinations and Methods of Using an Indolinone Compound

(75) Inventor: Charles M. Baum, San Diego, CA (US)

Correspondence Address: PFIZER INC, 10555 SCIENCE CENTER DRIVE, SAN DIEGO, CA, 92121, US

(73) Assignee: PFIZER INC., New York, NY (US)

(21) Appl. No.: 11/913,879

(22) PCT Filed: May 4, 2006
(86) PCT No.: PCT/IB06/01251

§ 371 Date: Apr. 15, 2008

Related U.S. Application Data
(60) Provisional application No. 60/680,837, filed on May 12, 2005.
(60) Provisional application No. 60/753,797, filed on Dec. 23, 2005.

(51) Int. Cl. A61K 39/395; A61K 31/4045; A61K 31/517; A61K 31/5377; A61K 31/513; A61K 31/565; A61K 31/675; A61K 31/4188; A61K 31/4196; A61P 35/00; A61K 31/4375; A61K 33/24; A61K 31/519

(52) U.S. Cl. 424/133.1; 514/414; 514/265.1; 424/145.1; 514/266.4; 424/649; 514/234.5; 514/283; 514/274; 514/255.05; 514/171; 514/383; 514/90; 514/393

(57) ABSTRACT

The invention provides methods of treating cancer using a compound of formula (1) or a pharmaceutically acceptable salt thereof, particularly a malate salt, in combination with various additional therapeutic agents. The invention also provides therapeutic dosing regimens, using the compound of formula (1) and an additional therapeutic agent.

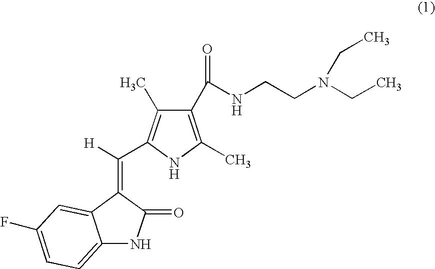

This application claims the benefit of U.S. Provisional Application Nos. 60/680,837, filed May 12, 2005 and 60/753,797, filed Dec. 23, 2005, the disclosures of which are incorporated by reference herein in their entireties.

### FIELD OF THE INVENTION

This invention relates to combinations and methods of treating abnormal cell growth, such as cancer, in mammals, particularly in humans. In particular, the invention provides combination therapies and treatment regimens for treatment of, for example, cancers, using an indolinone derivative that inhibits multiple receptor tyrosine kinases.

### BACKGROUND

The compound 5-(5-fluoro-2-oxo-1,2-dihydroindol-(3Z)-ylidenemethyl)-2,4-dimethyl-1H-pyrrole-3-carboxylic acid (2-diethylaminoethyl)-amide, represented by formula 1

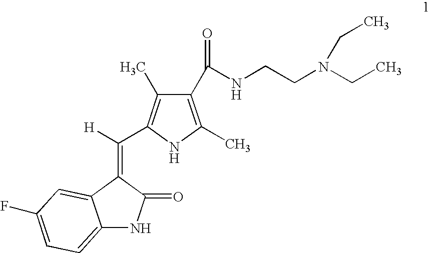

is a novel oral cancer drug shown to have efficacy in a variety of solid tumor types. Compound 1 targets multiple receptor tyrosine kinase inhibitors, including PDGFR, KIT and VEGFR, and is a potent and selective anti-angiogenesis agent. Compound 1 or its L-malate salt is also referred to as SU11248, SU011248, sunitinib malate (USAN/WHO designation) or SUTENT™ (L-malate salt).

The compound, its synthesis, and particular polymorphs are described in U.S. Pat. No. 6,573,293, U.S. Patent Publication Nos. 2003-0229229, 2003-0069298 and 2005-0059824, and in J. M. Manley, M. J. Kalman, B. G. Conway, C. C. Ball, J. L. Havens and R. Vaidyanathan, “Early Amidation Approach to 3-[(4-amido)pyrrol-2-yl]-2-indolinones,”J. Org. Chem. 68, 6447-6450 (2003). Preferred formulations of Compound 1 and its L-malate salt are described in PCT Publication No. WO 2004/024127. Preferred dosing regimens are described in U.S. patent application Ser. No. 10/991,244, filed Nov. 17, 2004, entitled “Method of Treating Abnormal Cell Growth Using Indolinone Compounds,” published as U.S. Patent Publication No. 2005-0182122. The disclosures of these references are incorporated herein by reference in their entireties.

Several references describe combinations of Compound 1 with other agents. For example, U.S. Patent Publication No. 2003-0216410 describes combinations of Compound 1 with cyclooxygenase inhibitors. U.S. Patent Publication No. 2004-0152759 describes combinations of Compound 1 with several agents, such as CPT-11 (irinotecan, Camptosar™), docetaxel and 5-fluorouracil (5-FU). U.S. Provisional Application Ser. Nos. 60/660,624, filed Mar. 11, 2005 and 60/664,653, both entitled “Anti-CTLA-4 Antibody and Indolinone Combination Therapy for Treatment of Cancer,” describe combinations of Compound 1 with an anti-CTLA-4 antibody. The disclosures of these references are incorporated herein by reference in their entireties.

It would be desirable to have additional combination therapies and treatment regimens using Compound 1, for the treatment of abnormal cell growth, such as cancers and ophthalmic disorders.

### SUMMARY

In one embodiment, the invention provides a method of treating cancer in a patient, comprising administering to the patient sunitinib malate in an amount of 25 to 75 mg free base equivalent daily, on a continuous dosing schedule.

In a particular aspect of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

In another particular aspect of this embodiment, the cancer is selected from the group consisting of a gastrointestinal stromal tumor, renal cell carcinoma, breast cancer, colorectal cancer, non-small cell lung cancer, a neuroendocrine tumor, thyroid cancer, small cell lung cancer, mastocytosis, glioma, sarcoma, acute myeloid leukemia, prostate cancer, lymphoma, and pancreatic cancer.

In another particular aspect of this embodiment, and in combination with any other particular aspect, the amount is 25, 37.5, 50 or 62.5 mg free base equivalent. In another particular aspect of this embodiment, and in combination with any other particular aspect, the amount is 75 mg free base equivalent.

In another embodiment, the invention provides a method of treating cancer in a patient, comprising administering to the patient sunitinib malate as an adjuvant therapy in an amount of 25 to 75 mg free base equivalent daily.

In a particular aspect of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

In another particular aspect of this embodiment, the cancer is selected from the group consisting of a gastrointestinal stromal tumor, renal cell carcinoma, breast cancer, colorectal cancer, non-small cell lung cancer, a neuroendocrine tumor, thyroid cancer, small cell lung cancer, mastocytosis, glioma, sarcoma, acute myeloid leukemia, prostate cancer, lymphoma, and pancreatic cancer.

In another particular aspect of this embodiment, sunitinib malate is administered on a continuous dosing schedule. In a further aspect, sunitinib malate is administered in an amount of 25, 37.5, 50 or 62.5 mg free base equivalent. In a further aspect, sunitinib malate is administered in an amount of 75 mg free base equivalent.

In another particular aspect of this embodiment, sunitinib malate is administered on an intermittent dosing schedule. In a further aspect, sunitinib malate is administered in an amount of 25, 37.5, 50 or 62.5 mg free base equivalent. In a further aspect, sunitinib malate is administered in an amount of 75 mg free base equivalent.

In another embodiment, the invention provides a method of treating cancer in a patient, comprising administering to the patient sunitinib malate as a neoadjuvant therapy in an amount of 25 to 75 mg free base equivalent daily.

In a particular aspect of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

In another particular aspect of this embodiment, the cancer is selected from the group consisting of a gastrointestinal stromal tumor, renal cell carcinoma, breast cancer, colorectal cancer, non-small cell lung cancer, a neuroendocrine tumor, thyroid cancer, small cell lung cancer, mastocytosis, glioma, sarcoma, acute myeloid leukemia, prostate cancer, lymphoma, and pancreatic cancer.

In another particular aspect of this embodiment, sunitinib malate is administered on a continuous dosing schedule. In a further aspect, sunitinib malate is administered in an amount of 25, 37.5, 50 or 62.5 mg free base equivalent. In a further aspect, sunitinib malate is administered in an amount of 75 mg free base equivalent.

In another particular aspect of this embodiment, sunitinib malate is administered on an intermittent dosing schedule. In a further aspect, sunitinib malate is administered in an amount of 25, 37.5, 50 or 62.5 mg free base equivalent. In a further aspect, sunitinib malate is administered in an amount of 75 mg free base equivalent.

In another embodiment, the invention provides a method of treating cancer in a patient, comprising administering to the patient sunitinib malate in an amount of 50 to 200 mg free base equivalent as a loading dose, followed by 25 to 75 mg free base equivalent daily, on an intermittent dosing schedule.

In a particular aspect of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

In another particular aspect of this embodiment, the cancer is selected from the group consisting of a gastrointestinal stromal tumor, renal cell carcinoma, breast cancer, colorectal cancer, non-small cell lung cancer, a neuroendocrine tumor, thyroid cancer, small cell lung cancer, mastocytosis, glioma, sarcoma, acute myeloid leukemia, prostate cancer, lymphoma, and pancreatic cancer.

In another particular aspect of this embodiment, and in combination with any other particular aspect, the amount of the loading dose is 50 or 100 or 150 or 200 mg free base equivalent. In another particular aspect of this embodiment, and in combination with any other particular aspect, the amount of the daily dose is 25, 37.5, 50, 62.5 or 75 mg free base equivalent. In another particular aspect of this embodiment, and in combination with any other particular aspect, the intermittent dosing schedule is a 2/1 or 2/2 schedule, wherein the dose on the first day of each treatment cycle is any of the loading doses described above, and the dose on the remaining dosing days of each treatment cycle is any of the daily doses described above. In a preferred aspect of this embodiment, the loading dose is 150 mg, the daily dose is 50 mg, and the dosing schedule is a 2/1 schedule.

In another embodiment, the invention provides a method of treating non-small cell lung cancer in a patient, comprising administering to the patient as a therapy following a first-line chemotherapy, sunitinib malate in an amount of 25 to 75 mg free base equivalent daily.

In a particular aspect of this embodiment, sunitinib malate is administered on a continuous dosing schedule. In a further aspect, sunitinib malate is administered in an amount of 25, 37.5, 50 or 62.5 mg free base equivalent. In a further aspect, sunitinib malate is administered in an amount of 75 mg free base equivalent.

In another particular aspect of this embodiment, sunitinib malate is administered on an intermittent dosing schedule. In a further aspect, sunitinib malate is administered in an amount of 25, 37.5, 50 or 62.5 mg free base equivalent. In a further aspect, sunitinib malate is administered in an amount of 75 mg free base equivalent.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating an angiogenesis- or VEGF-related ophthalmic disorder in a patient, comprising administering to the patient sunitinib malate in an amount of 25 to 75 mg free base equivalent daily.

In a particular aspect of this embodiment, the ophthalmic disorder is age related macular degeneration, choroidal neovascularization, retinopathy, retinitis, uveitis, retinal vein occlusion, iris neovascularization, corneal neovascularization, macular edema, or neovascular glaucoma.

In another embodiment, the invention provides a method of treating non-small cell lung cancer in a patient, comprising administering to the patient sunitinib malate in an amount of from 25 to 75 mg free base equivalent daily, and gefitinib daily in an amount of 250 mg.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating renal cell carcinoma in a patient, comprising administering to the patient sunitinib malate in an amount of from 25 to 75 mg free base equivalent daily, and gefitinib daily in an amount of 250 mg.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating non-small cell lung cancer in a patient, comprising administering to the patient sunitinib malate in an amount of from 25 to 50 mg free base equivalent daily, and erlotinib daily in an amount of 150 mg.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating renal cell carcinoma in a patient, comprising administering to the patient sunitinib malate in an amount of from 25 to 50 mg free base equivalent daily, and erlotinib daily in an amount of 150 mg.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating pancreatic cancer in a patient, comprising administering to the patient sunitinib malate in an amount of from 25 to 50 mg free base equivalent daily, and erlotinib daily in an amount of 150 mg.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating colorectal cancer in a patient, comprising administering to the patient sunitinib malate in an amount of from 25 to 50 mg free base equivalent daily, and cetuximab in an initial infusion of 400 mg/m² followed by weekly infusions of 250 mg/m².

In a further aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating head and neck cancer in a patient, comprising administering to the patient sunitinib malate in an amount of from 25 to 50 mg free base equivalent daily, and cetuximab in an initial infusion of 400 mg/m² followed by weekly infusions of 250 mg/m².

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating breast cancer in a patient, comprising administering to the patient sunitinib malate in an amount of from 25 to 50 mg free base equivalent daily, and docetaxel in an infusion of 60 to 100 mg/m² once every three weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating non-small cell lung cancer in a patient, comprising administering to the patient sunitinib malate in an amount of from 25 to 50 mg free base equivalent daily, and docetaxel in an infusion of 60 to 100 mg/m² once every three weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating prostate cancer in a patient, comprising administering to the patient sunitinib malate in an amount of from 25 to 50 mg free base equivalent daily, and docetaxel in an infusion of 60 to 100 mg/m² once every three weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating pancreatic cancer in a patient, comprising administering to the patient sunitinib malate in an amount of from 25 to 50 mg free base equivalent daily, and gemcitabine in an infusion of 750 to 1250 mg/m² once weekly on a 4/1, 3/1 or 2/1 weekly dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating non-small cell cancer in a patient, comprising administering to the patient sunitinib malate in an amount of from 25 to 50 mg free base equivalent daily, and gemcitabine in an infusion of 750 to 1250 mg/m² once weekly on a 4/1, 3/1 or 2/1 weekly dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 3/1 or a 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating bladder cancer in a patient, comprising administering to the patient sunitinib malate in an amount of from 25 to 50 mg free base equivalent daily, and gemcitabine in an infusion of 750 to 1250 mg/m² once weekly on a 4/1, 3/1 or 2/1 weekly dosing schedule.

In a particular aspect of this embodiment, sunitinib malate is administered on a continuous dosing schedule.

In a particular aspect of this embodiment, sunitinib malate is administered on an intermittent dosing schedule. In a further aspect, the intermittent dosing schedule is a 4/2 or 3/1 or 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating breast cancer in a patient, comprising administering to the patient sunitinib malate in an amount of from 25 to 50 mg free base equivalent daily, and capecitabine in an amount of 825 to 1250 mg/m² twice daily on a 2/1 dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating colorectal cancer in a patient, comprising administering to the patient sunitinib malate in an amount of from 25 to 50 mg free base equivalent daily, and capecitabine in an amount of 825 to 1250 mg/m² twice daily on a 2/1 dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating colorectal cancer in a patient, comprising administering to the patient sunitinib malate in an amount of from 25 to 50 mg free base equivalent daily, and oxaliplatin, 5-fluorouracil and leucovorin on a FOLFOX4 dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 2/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating colorectal cancer in a patient, comprising administering to the patient sunitinib malate in an amount of from 25 to 50 mg free base equivalent daily, and irinotecan, 5-fluorouracil and leucovorin on a FOLFIRI dosing schedule.

In a particular aspect of this embodiment, sunitinib malate is administered on a continuous dosing schedule.

In a particular aspect of this embodiment, sunitinib malate is administered on an intermittent dosing schedule. In a further aspect, the intermittent dosing schedule is a 4/2 or a 2/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating gastrointestinal stromal tumors (GIST) in a patient, comprising administering to the patient sunitinib malate in an amount of from 25 to 50 mg free base equivalent daily, and imatinib once daily in an amount of 400 to 600 mg on a continuous dosing schedule.

In a particular aspect of this embodiment, sunitinib malate is administered on a continuous dosing schedule.

In a particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating HER2 positive breast cancer in a patient, comprising administering to the patient sunitinib malate in an amount of from 25 to 50 mg free base equivalent daily, and trastuzumab on a once weekly dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating non-small cell lung cancer in a patient, comprising administering to the patient sunitinib malate in an amount of from 25 to 50 mg free base equivalent daily, and pemetrexed in an amount of 250 to 500 mg/m² once every three weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating breast cancer in a patient, comprising administering to the patient sunitinib malate in an amount of from 25 to 50 mg free base equivalent daily, and exemestane in an amount of 25 mg once daily.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating breast cancer in a patient, comprising administering to the patient sunitinib malate in an amount of from 25 to 50 mg free base equivalent daily, and anastrozole in an amount of 1 mg once daily.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating breast cancer in a patient, comprising administering to the patient sunitinib malate in an amount of from 25 to 50 mg free base equivalent daily, and letrozole in an amount of 2.5 mg once daily.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating brain tumors in a patient, comprising administering to the patient sunitinib malate in an amount of from 25 to 50 mg free base equivalent daily, and temozolomide once daily in an amount of 150 to 200 mg/m² on the first 5 days of a 4-week dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or 3/1 or 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating melanoma in a patient, comprising administering to the patient sunitinib malate in an amount of from 25 to 50 mg free base equivalent daily, and temozolomide once daily in an amount of 150 to 200 mg/m² on the first 5 days of a 4-week dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or 3/1 or 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating melanoma in a patient, comprising administering to the patient sunitinib malate in an amount of from 25 to 50 mg free base equivalent daily, and dacarbazine in an amount of 2 to 4.5 mg/kg/day on the first 10 days only of a 4-week treatment cycle or in an amount of 250 mg/m²/day on the first 5 days of a 3-week treatment cycle.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or 3/1 or 2/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating renal cell carcinoma in a patient, comprising administering to the patient sunitinib malate in an amount of from 25 to 50 mg free base equivalent daily, and bevacizumab in an amount of 3 to 10 mg/kg once every two weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating colorectal cancer in a patient, comprising administering to the patient sunitinib malate in an amount of from 25 to 50 mg free base equivalent daily, and bevacizumab in an amount of 3 to 10 mg/kg once every two weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating non-small cell lung cancer in a patient, comprising administering to the patient sunitinib malate in an amount of from 25 to 50 mg free base equivalent daily, and bevacizumab in an amount of 3 to 10 mg/kg once every two weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating prostate cancer in a patient, comprising administering to the patient sunitinib malate in an amount of from 25 to 50 mg free base equivalent daily, and bevacizumab in an amount of 3 to 10 mg/kg once every two weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating breast cancer in a patient, comprising administering to the patient sunitinib malate in an amount of from 25 to 50 mg free base equivalent daily, and bevacizumab in an amount of 3 to 10 mg/kg once every two weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In another embodiment, the invention provides a method of treating a sarcoma in a patient, comprising administering to the patient sunitinib malate in an amount of from 25 to 50 mg free base equivalent daily, and doxorubicin in an amount of 40 to 75 mg/m² once every 3 or 4 weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 3/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating a sarcoma in a patient, comprising administering to the patient sunitinib malate in an amount of from 25 to 50 mg free base equivalent daily, and epirubicin in an amount of 60 to 120 mg/m² once every 3 or 4 weeks.

In a particular aspect of this embodiment, sunitinib malate is administered on a continuous dosing schedule.

In a particular aspect of this embodiment, sunitinib malate is administered on an intermittent dosing schedule. In a further aspect, the intermittent dosing schedule is a 4/2 or a 3/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating non-small cell lung cancer in a patient, comprising administering to the patient sunitinib malate in an amount of from 25 to 50 mg free base equivalent daily, paclitaxel in an amount of 135 to 175 mg/m² once every 3 weeks, and carboplatin in an amount sufficient to achieve a target AUC concentration of 4 to 7 mg/mL/min once every three weeks following the paclitaxel dose.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating ovarian cancer in a patient, comprising administering to the patient sunitinib malate in an amount of from 25 to 50 mg free base equivalent daily, paclitaxel in an amount of 135 to 175 mg/m² once every 3 weeks, and carboplatin in an amount sufficient to achieve a target AUC concentration of 4 to 7 mg/mL/min once every three weeks following the paclitaxel dose.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating breast cancer in a patient, comprising administering to the patient sunitinib malate in an amount of from 25 to 50 mg free base equivalent daily, preferably 25, 37.5 or 50 mg; and paclitaxel in an amount of 50 to 175 mg/m² once weekly for 3 weeks followed by a one-week paclitaxel rest period (i.e., no paclitaxel dose on week 4), preferably 65 or 90 mg/m² once weekly for 3 weeks followed by a one-week paclitaxel rest period.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 3/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating non-small cell lung cancer in a patient, comprising administering to the patient sunitinib malate in an amount of from 25 to 50 mg free base equivalent daily; gemcitabine in an amount of 750 to 1250 mg/m² once weekly for 2, 3 or 4 weeks followed by a one-week rest period; and cisplatin in an amount of 50 to 100 mg/m² once every 3 or 4 weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating bladder cancer in a patient, comprising administering to the patient sunitinib malate in an amount of from 25 to 50 mg free base equivalent daily; gemcitabine in an amount of 750 to 1250 mg/m² once weekly for 2, 3 or 4 weeks followed by a one-week rest period; and cisplatin in an amount of 50 to 100 mg/m² once every 3 or 4 weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating breast cancer in a patient, comprising administering to the patient sunitinib malate in an amount of from 25 to 50 mg free base equivalent daily; doxorubicin in an amount of 40 to 75 mg/m² once every 3 or 4 weeks; and cyclophosphamide in an amount of 400 to 800 mg/m² once every 3 or 4 weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating breast cancer in a patient, comprising administering to the patient sunitinib malate in an amount of from 25 to 50 mg free base equivalent daily; 5-fluorouracil on an intermittent dosing schedule; epirubicin in an amount of 60 to 120 mg/m² once every 3 or 4 weeks; and cyclophosphamide in an amount of 400 to 800 mg/m² once every 3 or 4 weeks.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 2/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating HER2 positive breast cancer in a patient, comprising administering to the patient sunitinib malate in an amount of from 25 to 50 mg free base equivalent daily; paclitaxel in an amount of 135 to 175 mg/m² once every 3 weeks; and trastuzumab in an amount 2 mg/kg once weekly.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 2/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating colorectal cancer in a patient, comprising administering to the patient sunitinib malate in an amount of from 25 to 50 mg free base equivalent daily; and irinotecan, 5-fluorouracil and leucovorin in an IFL dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating colorectal cancer in a patient, comprising administering to the patient sunitinib malate in an amount of from 25 to 50 mg free base equivalent daily; and a MEK inhibitor.

In a particular aspect of this embodiment, the MEK inhibitor is N—[(R)-2,3-dihydroxy-propoxy]-3,4-difluoro-2-(2-fluoro-4-iodo-phenylamino)-benzamide, or a pharmaceutically acceptable salt thereof. In a further aspect, the MEK inhibitor is administered in an amount of 10 to 30 mg once or twice daily on a continuous dosing schedule.

In another particular aspect of this embodiment, and in combination with any other particular aspect not inconsistent, sunitinib malate is administered on a continuous dosing schedule.

In another particular aspect of this embodiment, and in combination with any other particular aspect not inconsistent, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating prostate cancer in a patient, comprising administering to the patient sunitinib malate in an amount of from 25 to 50 mg free base equivalent daily; docetaxel in an amount of 75 mg/m² once every three weeks; and prednisone in an amount of 5 mg twice daily on a continuous dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 4/2 or a 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating prostate cancer in a patient, comprising administering to the patient sunitinib malate in an amount of from 25 to 50 mg free base equivalent daily; and an anti-androgen on a continuous dosing schedule.

In a particular aspect of this embodiment, the anti-androgen is selected from the group consisting of bicalutamide, flutamide, and nilutamide.

In another particular aspect of this embodiment, and in combination with any other particular aspect not inconsistent, sunitinib malate is administered on a continuous dosing schedule.

In another particular aspect of this embodiment, and in combination with any other particular aspect not inconsistent, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating prostate cancer in a patient, comprising administering to the patient sunitinib malate in an amount of from 25 to 50 mg free base equivalent daily; and an LHRH agonist or antagonist.

In a particular aspect of this embodiment, the LHRH agonist is leuprolide or goserelin.

In another particular aspect of this embodiment, the LHRH antagonist is abarelix.

In another particular aspect of this embodiment, and in combination with any other particular aspect not inconsistent, sunitinib malate is administered on a continuous dosing schedule.

In another particular aspect, and in combination with any other particular aspect not inconsistent, sunitinib malate is administered on an intermittent dosing schedule. In a further aspect, the intermittent dosing schedule is a 4/2 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a-daily dose of 25 to 75 mg free base equivalent on the remaining days of each treatment cycle.

In another embodiment, the invention provides a method of treating prostate cancer in a patient, comprising administering to the patient sunitinib malate in an amount of from 12.5 to 50 mg free base equivalent daily, preferably 12.5, 25, 37.5 or 50 mg; docetaxel in an amount of 60 mg/m2 every three weeks; and prednisone in an amount of 5 mg twice daily on a continuous dosing schedule.

In a particular aspect of this embodiment, sunitinib malate is administered on a continuous dosing schedule.

In another particular aspect of this embodiment, sunitinib malate is administered on an intermittent dosing schedule. In a further aspect, the intermittent dosing schedule is a 2/1 dosing schedule.

In a another particular aspect of this embodiment, sunitinib malate is administered on an intermittent dosing schedule, particularly a 2/1 or 2/2 schedule, in a loading dose of 50 to 200 mg free base equivalent on the first day of each treatment cycle, and a daily dose of 25 to 75 mg free base equivalent on the remaining days of each treatment cycle.

In another embodiment, the invention provides a method of treating an ocular neovascular disorder in a patient in need thereof, by administering to the patient a therapeutically effective amount of compound I. In a particular aspect of this embodiment, the ocular neovascular disorder is age-related macular degeneration, choroidal neovascularization, a retinopathy, retinitis, uveitis, retinal vein occlusion, iris neovascularization, corneal neovascularization, macular edema, or neovascular glaucoma. In a further aspect of this embodiment, the retinopathy is diabetic retinopathy, vitreoretinopathy or retinopathy of prematurity.

A “pharmaceutical composition” refers to a mixture of one or more of the compounds described herein, or physiologically/pharmaceutically acceptable salts, solvates, hydrates or prodrugs thereof, with other chemical components, such as physiologically/pharmaceutically acceptable carriers and excipients. The purpose of a pharmaceutical composition is to facilitate administration of a compound to an organism.

As used herein, a “physiologically/pharmaceutically acceptable carrier” refers to a carrier or diluent that does not cause significant irritation to an organism and does not abrogate the biological activity and properties of the administered compound.

A “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “pharmaceutically acceptable salt” refers to those salts which retain the biological effectiveness and properties of the parent compound. Such salts include acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like.

### DETAILED DESCRIPTION

As used herein, unless otherwise indicated, the term “Compound 1” refers to the compound of structural formula 1

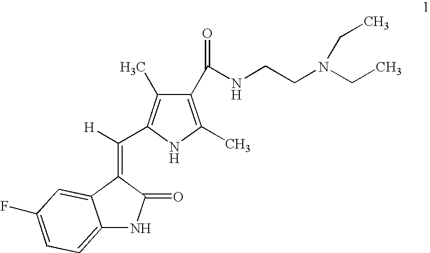

also designated as 5-(5-fluoro-2-oxo-1,2-dihydroindol-(3Z)-ylidenemethyl)-2,4-dimethyl-1H-pyrrole-3-carboxylic acid (2-diethylaminoethyl)-amide, in its free base form, as well as pharmaceutically acceptable salts or solvates (including hydrates) thereof. A particularly preferred salt is a malate salt, more preferably the L-malate salt.

References to amounts of Compound 1 refer to free base equivalent amounts. For example, if Compound 1 is used in the form of a salt, reference to “50 mg of Compound 1” or “50 mg of Compound 1, free base equivalent” means the amount of salt that would be needed to provide 50 mg of the free base upon complete dissociation of the salt. For the specific example of the malate salt, 50 mg of Compound 1, free base equivalent, is provided by 66.6 mg of the malate salt.

Specific dosing guidance is given for various indications and combinations. However, the following general comments apply unless otherwise indicated.

Compound 1 is conveniently provided as an oral dosage form, such as a tablet or capsule, in dosage amounts of 12.5, 25 or 50 mg, free base equivalent. These dosage amounts permit easy dosing adjustments in 12.5 mg increments. Although other dosage amounts are possible, typical dosing ranges are from 12.5 to 75 mg per day, and more typically 25, 37.5, 50 or 62.5 mg per day, free base equivalent. The daily dose is generally taken at a frequency of once per day, without regard to food; i.e., in a fed or fasted state. Compound 1 can be administered in a continuous dosing regimen, i.e., daily for the duration of the treatment period, or in an intermittent dosing regimen, i.e., administered daily during a treatment period, followed by a rest or non-treatment period during which no Compound 1 is administered. In an intermittent dosing regimen, the treatment period is typically from 10 to 30 days, such as 2, 3 or 4 weeks, and the rest period is typically from 3 to 15 days, such as 1 or 2 weeks. The combination of any treatment period from 10 to 30 days with any rest period from 3 to 15 days is contemplated. Several intermittent regimens are presently preferred; expressed as treatment period in weeks/rest period in weeks, preferred regimens include 4/2, 4/1, 3/2, 3/1 and 2/1. Other intermittent regimens include a loading dose on the first day of each treatment cycle, and a lower daily dose on each remaining dosing day of each treatment cycle. It should be further appreciated that dosing regimens can be adjusted by one skilled in the art to more conveniently accommodate coordination of the dosing regimens of Compound 1 and additional therapeutic agents, if such adjustments are therapeutically acceptable. For example, if an additional therapeutic agent were administered as an infusion once every 4 weeks, a Compound 1 dosing regimen of 3/1 or 2/2, or a continuous dosing regimen, would best coordinate with the regimen of the additional therapeutic agent.

Compound 1 is useful in the treatment of abnormal cell growth, such as cancer, including, but not limited to, lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In a particular aspect of this embodiment, the cancer is selected from gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, and combinations thereof.

In another embodiment, the abnormal cell growth is a benign proliferative disease, including, but not limited to, psoriasis, benign prostatic hypertrophy or restinosis.

Compound 1 is also useful in the treatment of angiogenesis- or VEGF-related ophthalmic disorders, or ocular neovascular disorders, such as age related macular degeneration (ARMD), choroidal neovascularization (CNV), retinopathies (e.g., diabetic retinopathy, vitreoretinopathy, retinopathy of prematurity), retinitis (e.g., cytomegalovirus (CMV retinitis), uveitis, retinal vein occlusion, iris neovascularization, corneal neovascularization, macular edema, and neovascular glaucoma. A detailed discussion of such disorders can be found in A. Adamis and D. Shima, “The Role of Vascular Endothethial Growth Factor in Ocular Health and Disease”, Retina, 25:111-118, 2005, the disclosure of which is incorporated herein by reference in its entirety.

In a particular embodiment, the invention provides a method of treating any of the above-recited cancers in a patient, such as a human, by administering to the patient Compound 1 in an amount of 25 to 75 mg, preferably 37.5, 50 or 62.5 mg, daily, on a continuous (i.e., not intermittent) dosing schedule. One skilled in the art can readily determine the optimal dosage for a particular patient based on tumor response and adverse event profile.

In a particular aspect of this embodiment, the cancer is a gastrointestinal stromal tumor.

In another particular aspect of this embodiment, the cancer is renal cell carcinoma.

In another particular aspect of this embodiment, the cancer is breast cancer.

In another particular aspect of this embodiment, the cancer is colorectal cancer.

In another particular aspect of this embodiment, the cancer is non-small cell lung cancer.

In another particular aspect of this embodiment, the cancer is a neuroendocrine tumor.

In another particular aspect of this embodiment, the cancer is thyroid cancer.

In another particular aspect of this embodiment, the cancer is small cell lung cancer.

In another particular aspect of this embodiment, the cancer is mastocytosis.

In another particular aspect of this embodiment, the cancer is glioma.

In another particular aspect of this embodiment, the cancer is sarcoma.

In another particular aspect of this embodiment, the cancer is acute myeloid leukemia.

In another particular aspect of this embodiment, the cancer is prostate cancer.

In another particular aspect of this embodiment, the cancer is lymphoma.

In another particular aspect of this embodiment, the cancer is pancreatic cancer.

In a particular embodiment, the invention provides a method of treating any of the above-recited cancers in a patient, such as a human, by administering to the patient Compound 1 as an adjuvant therapy in an amount of 25 to 75 mg, preferably 37.5, 50 or 62.5 mg, daily, on a continuous (i.e., not intermittent) or intermittent dosing schedule. As used herein, the term “adjuvant therapy” refers to treatment after surgical resection of the primary tumor. One skilled in the art can readily determine the optimal dosage for a particular patient based on tumor response and adverse event profile.

In a particular aspect of this embodiment, the cancer is a gastrointestinal stromal tumor.

In another particular aspect of this embodiment, the cancer is renal cell carcinoma.

In another particular aspect of this embodiment, the cancer is breast cancer.

In another particular aspect of this embodiment, the cancer is colorectal cancer.

In another particular aspect of this embodiment, the cancer is non-small cell lung cancer.

In another particular aspect of this embodiment, the cancer is a neuroendocrine tumor.

In another particular aspect of this embodiment, the cancer is thyroid cancer.

In another particular aspect of this embodiment, the cancer is small cell lung cancer.

In another particular aspect of this embodiment, the cancer is mastocytosis.

In another particular aspect of this embodiment, the cancer is glioma.

In another particular aspect of this embodiment, the cancer is sarcoma.

In another particular aspect of this embodiment, the cancer is acute myeloid leukemia.

In another particular aspect of this embodiment, the cancer is prostate cancer.

In another particular aspect of this embodiment, the cancer is lymphoma.

In another particular aspect of this embodiment, the cancer is pancreatic cancer.

In a particular embodiment, the invention provides a method of treating any of the above-recited cancers in a patient, such as a human, by administering to the patient Compound 1 as a neoadjuvant therapy in an amount of 25 to 75 mg, preferably 37.5, 50 or 62.5 mg, daily, on a continuous (i.e., not intermittent) or intermittent dosing schedule. As used herein, the term “neoadjuvant therapy” refers to treatment prior to the surgical resection of a primary malignant tumor. One skilled in the art can readily determine the optimal dosage for a particular patient based on tumor response and adverse event profile.

In a particular aspect of this embodiment, the cancer is a gastrointestinal stromal tumor.

In another particular aspect of this embodiment, the cancer is renal cell carcinoma.

In another particular aspect of this embodiment, the cancer is breast cancer, particularly stage IIA, stage IIB or stage III breast cancer. Stage IIA breast cancer is either no larger than 2 cm and has spread to the axillary lymph nodes, or between 2 and 5 cm but has not spread to the axillary lymph nodes. Stage IIB breast cancer is either between 2 and 5 cm and has spread to the axillary lymph nodes, or larger than 5 cm but has not spread to the axillary lymph nodes. Stage III breast cancer is a primary cancer that measures less than 5 cm in size and causes axillary lymph nodes to be attached to each other or other structures; a primary cancer that is greater than 5 cm and involves axillary lymph nodes; or a primary cancer that is attached to the chest wall or skin. Neoadjuvant therapy in breast cancer is described more fully in G. F. Schwartz et al., “Proceedings of the Consensus Conference on Neoadjuvant Chemotherapy in Carcinoma of the Breast, Apr. 26-28, 2003, Philadelphia, Pa.,” Cancer, Jun. 15, 2004, vol. 100: 2512-2532.

In another particular aspect of this embodiment, the cancer is colorectal cancer.

In another particular aspect of this embodiment, the cancer is non-small cell lung cancer.

In another particular aspect of this embodiment, the cancer is a neuroendocrine tumor.

In another particular aspect of this embodiment, the cancer is thyroid cancer.

In another particular aspect of this embodiment, the cancer is small cell lung cancer.

In another particular aspect of this embodiment, the cancer is mastocytosis.

In another particular aspect of this embodiment, the cancer is glioma.

In another particular aspect of this embodiment, the cancer is sarcoma.

In another particular aspect of this embodiment, the cancer is acute myeloid leukemia.

In another particular aspect of this embodiment, the cancer is prostate cancer.

In another particular aspect of this embodiment, the cancer is lymphoma.

In another particular aspect of this embodiment, the cancer is pancreatic cancer.

In a particular embodiment, the invention provides a method of treating non-small cell lung cancer in a patient, such as a human, by administering to the patient as a therapy following a first-line chemotherapy, Compound 1 in an amount of 25 to 75 mg, preferably 37.5, 50 or 62.5 mg, daily, on a continuous or intermittent dosing schedule. One skilled in the art can readily determine the optimal dosage for a particular patient based on response and adverse event profile. In this embodiment, the first line chemotherapy can be any of the first line chemotherapy regimens well know in the art for non-small cell lung cancer, such as, for example, regimens described in C. P. Belani and C. Langer, “First-line chemotherapy for NSCLC: an overview of relevant trials,” Lung Cancer 38, S13-S19 (2002).

In a particular embodiment, the invention provides a method of treating any of the above-recited benign proliferative diseases in a patient, such as a human, by administering to the patient Compound 1 in an amount of 25 to 75 mg, preferably 37.5, 50 or 62.5 mg, daily, on a continuous (i.e., not intermittent) dosing schedule. One skilled in the art can readily determine the optimal dosage for a particular patient based on response and adverse event profile.

In a particular embodiment, the invention provides a method of treating any of the above-recited angiogenesis or VEGF-related ophthalmic disorders, or ocular neovascular disorders, in a patient, such as a human, by administering to the patient Compound 1 in an amount of 25 to 75 mg, preferably 37.5, 50 or 62.5 mg, daily, on a continuous or intermittent dosing schedule. One skilled in the art can readily determine the optimal dosage for a particular patient based on response and adverse event profile.

### Combination Therapies

The invention also provides combinations, methods of using combinations and kits for use in combination therapies, using Compound 1 and a variety of other agents.

In one embodiment, the invention is directed to combination treatments using Compound 1 and an EGFR inhibitor. Compound 1 can be administered in the dosage amounts and schedules described herein. Suitable EGFR inhibitors include gefitinib (Iressa™, AstraZeneca), erlotinib (Tarceva™ or OSI-774, OSI Pharmaceuticals Inc.), cetuximab (Erbitux™, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof. Preferred EGFR inhibitors include gefitinib, erlotinib and cetuximab, and combinations thereof.

(1) Iressa™/gefitinib: In one embodiment, the EGFR inhibitor is gefitinib, available from AstraZeneca as a 250 mg tablet under the tradename Iressa™. Preferably, the combination is used to treat a patient, preferably a human, suffering from cancer. Thus, in a particular aspect of this embodiment, the invention provides a method of treating cancer by administering to a patient Compound 1 and gefitinib in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Gefitinib can be administered in a dosage of from 250 to 500 mg once daily, preferably 250 mg once daily orally. Compound 1 and gefitinib can be administered at the same time, or sequentially, without regard to order. Compound 1 and gefitinib can be administered continuously (i.e., daily for the duration of the treatment). More preferably, Compound 1 is administered in an intermittent dosing regimen, and both gefitinib and Compound 1 are administered continuously during the Compound 1 treatment period, and only gefitinib is administered in the Compound 1 rest period. Both compounds can be administered in a fed or fasted state. In a particularly preferred embodiment, Compound 1 is administered in an amount of 25 to 50, preferably 25, 37.5 or 50 mg daily, on a 4/2 dosing schedule, and gefitinib is administered once daily in an amount of 250 mg. In another particularly preferred embodiment, Compound 1 is administered in an amount of 25 to 50, preferably 25, 37.5 or 50 mg daily, on a continuous dosing schedule, and gefitinib is administered once daily in an amount of 250 mg.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a 4/2 dosing schedule, and gefitinib in an amount of 250 mg daily on a continuous schedule.

In another particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a continuous dosing schedule, and gefitinib in an amount of 250 mg daily on a continuous schedule.

In another particularly preferred aspect of this embodiment, the invention provides a method of treating renal cell carcinoma in a patient, such as a human, by administering to the patient Compound 1 in an amount of 25 to 50, preferably 25, 37.5 or 50 mg daily, on a 4/2 dosing schedule, and gefitinib in an amount of 250 mg daily on a continuous schedule.

In another particularly preferred aspect of this embodiment, the invention provides a method of treating renal cell carcinoma in a patient, such as a human, by administering to the patient Compound 1 in an amount of 25 to 50, preferably 25, 37.5 or 50 mg daily, on a continuous dosing schedule, and gefitinib in an amount of 250 mg daily on a continuous schedule.

(2) Tarceva™/erlotinib: In another embodiment, the EGFR inhibitor is erlotinib, available from OSI Pharmaceuticals as a 25, 100 or 150 mg tablet under the tradename Tarceva™. Preferably, the combination is used to treat a patient, preferably a human, suffering from cancer. Thus, in a particular aspect of this embodiment, the invention provides a method of treating cancer by administering to a patient Compound 1 and erlotinib in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Erlotinib can be administered in a dosage of from 100 to 200 mg once daily, preferably 150 mg once daily orally. Compound 1 and erlotinib can be administered at the same time, or sequentially, without regard to order. Compound 1 and erlotinib can be administered continuously (i.e., daily for the duration of the treatment). More preferably, Compound 1 is administered in an intermittent dosing regimen, and both erlotinib and Compound 1 are administered continuously during the Compound 1 treatment period, and only erlotinib is administered in the Compound 1 rest period. Compound 1 can be administered in a fed or fasted state, but erlotinib is preferably administered in a fasted state, i.e., between meals, at least 1 hour before, or at least 2 hours after, a meal. In a particularly preferred embodiment, Compound 1 is administered in an amount of 25 to 50, preferably 25, 37.5 or 50 mg daily, on a 4/2 dosing schedule, and erlotinib is administered once daily in an amount of 150 mg. In another particularly preferred embodiment, Compound 1 is administered in an amount of 25 to 50, preferably 25, 37.5 or 50 mg daily, on a continuous dosing schedule, and erlotinib is administered once daily in an amount of 150 mg.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a 4/2 dosing schedule, and erlotinib in an amount of 150 mg daily on a continuous schedule.

In another particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a continuous dosing schedule, and erlotinib in an amount of 150 mg daily on a continuous schedule.

In another particularly preferred aspect of this embodiment, the invention provides a method of treating renal cell carcinoma in a patient, such as a human, by administering to the patient Compound 1 in an amount of 25 to 50, preferably 25, 37.5 or 50 mg daily, on a 4/2 dosing schedule, and erlotinib in an amount of 150 mg daily on a continuous schedule.

In another particularly preferred aspect of this embodiment, the invention provides a method of treating renal cell carcinoma in a patient, such as a human, by administering to the patient Compound 1 in an amount of 25 to 50, preferably 25, 37.5 or 50 mg daily, on a continuous dosing schedule, and erlotinib in an amount of 150 mg daily on a continuous schedule.

In another particularly preferred aspect of this embodiment, the invention provides a method of treating pancreatic cancer in a patient, such as a human, by administering to the patient Compound 1 in an amount of 25 to 50, preferably 25, 37.5 or 50 mg daily, on a 4/2 dosing schedule, and erlotinib in an amount of 150 mg daily on a continuous schedule.

In another particularly preferred aspect of this embodiment, the invention provides a method of treating pancreatic cancer in a patient, such as a human, by administering to the patient Compound 1 in an amount of 25 to 50, preferably 25, 37.5 or 50 mg daily, on a continuous dosing schedule, and erlotinib in an amount of 150 mg daily on a continuous schedule.

(3) Erbitux™/cetuximab: In another embodiment, the EGFR inhibitor is cetuximab, available from ImClone Systems Inc. in a single-use, 50 mL vial containing 100 mg of the compound in a sterile, injectable liquid, under the tradename Erbitux™. Preferably, the combination is used to treat a patient, preferably a human, suffering from cancer. Thus, in a particular aspect of this embodiment, the invention provides a method of treating cancer by administering to a patient Compound 1 and cetuximab in-amounts that in combination are therapeutically effective. In this embodiment, Compound 1 can be administered in a dosage of from 25 to 75 mg once daily, preferably 25, 37.5, 50, 62.5 or 75 mg once daily, preferably orally. Cetuximab can be administered in an initial loading dose of 400 mg/m² as a 120-minute intravenous infusion followed by weekly maintenance doses of from 150 to 250 mg/m², preferably 250 mg/m², infused over 60 minutes. Compound 1 and cetuximab can be administered at the same time, or sequentially, without regard to order. Compound 1 can be administered continuously (i.e., daily for the duration of the treatment), or more preferably, in an intermittent dosing regimen, and cetuximab can be administered once weekly without regard to Compound 1 dosing schedule. Both Compound 1 and cetuximab can be administered in a fed or fasted state. In a particularly preferred embodiment, Compound 1 is administered in an amount of 25 to 50, preferably 25, 37.5 or 50 mg daily, on a 4/2 dosing schedule, and cetuximab is administered in an initial infusion of 400 mg/m² followed by weekly infusions of 250 mg/m². In another particularly preferred embodiment, Compound 1 is administered in an amount of 25 to 50, preferably 25, 37.5 or 50 mg daily, on a continuous dosing schedule, and cetuximab is administered in an initial infusion of 400 mg/m² followed by weekly infusions of 250 mg/m².

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a 4/2 dosing schedule, and cetuximab in an initial infusion of 400 mg/m² followed by weekly infusions of 250 mg/m².

In another particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a continuous dosing schedule, and cetuximab in an initial infusion of 400 mg/m² followed by weekly infusions of 250 mg/m².

In another particularly preferred aspect of this embodiment, the invention provides a method of treating head and neck cancer in a patient, such as a human, by administering to the patient Compound 1 in an amount of 25 to 50, preferably 25, 37.5 or 50 mg daily, on a 4/2 dosing schedule, and cetuximab in an initial infusion of 400 mg/m² followed by weekly infusions of 250 mg/m².

In another particularly preferred aspect of this embodiment, the invention provides a method of treating head and neck cancer in a patient, such as a human, by administering to the patient Compound 1 in an amount of 25 to 50, preferably 25, 37.5 or 50 mg daily, on a continuous dosing schedule, and cetuximab in an initial infusion of 400 mg/m² followed by weekly infusions of 250 mg/m².

(4) Taxotere™/docetaxel: In another embodiment, the invention provides combination therapies of Compound 1 and docetaxel, an antineoplastic agent available from Aventis Pharmaceuticals as an injection concentrate in single-use vials containing 20 mg (0.5 mL) or 80 mg (2 mL) docetaxel (anhydrous), under the tradename Taxotere™. Preferably, the combination is used to treat a patient, preferably a human, suffering from cancer. Thus, in a particular aspect of this embodiment, the invention provides a method of treating cancer by administering to a patient Compound 1 and docetaxel in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Docetaxel, diluted for infusion as directed by the manufacturer can be administered in a dosage of from 60 to 100 mg/m², preferably 60, 75 or 100 mg/m², as a 60-minute intravenous infusion once every three weeks. Compound 1 and docetaxel can be administered at the same time, or sequentially, without regard to order. Compound 1 can be administered continuously (i.e., daily for the duration of the treatment), or more preferably, in an intermittent dosing regimen, and docetaxel can be administered once every three weeks without regard to Compound 1 dosing schedule. Both Compound 1 and docetaxel can be administered in a fed or fasted state. In a particularly preferred embodiment, Compound 1 is administered in an amount of 25 to 50, preferably 25, 37.5 or 50 mg daily, on a 4/2 or 2/1 dosing schedule, and docetaxel is administered in an infusion of 60, 75 or 100 mg/m² once every three weeks. In another particularly preferred embodiment, Compound 1 is administered in an amount of 25 to 50, preferably 25, 37.5 or 50 mg daily, on a continuous dosing schedule, and docetaxel is administered in an infusion of 60 to 100 mg/m² once every three weeks.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating breast cancer in a patient, such as a human, by administering to the patient Compound 1 in an amount of 25 to 50, preferably 25, 37.5 or 50 mg daily, on a 4/2 or 2/1 dosing schedule, and docetaxel in an infusion of 60, 75 or 100 mg/m² once every three weeks.

In another particularly preferred aspect of this embodiment, the invention provides a method of treating breast cancer in a patient, such as a human, by administering to the patient Compound 1 in an amount of 25 to 50, preferably 25, 37.5 or 50 mg daily, on a continuous dosing schedule, and docetaxel in an infusion of 60, 75 or 100 mg/m² once every three weeks.

In another particularly preferred aspect of this embodiment; the invention provides a method of treating non-small cell lung cancer in a patient, such as a human, by administering to the patient Compound 1 in an amount of 25 to 50, preferably 25, 37.5 or 50 mg daily, on a 4/2 or 2/1 dosing schedule, and docetaxel in an infusion of 60, 75 or 100 mg/m² once every three weeks.

In another particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a continuous dosing schedule, and docetaxel in an infusion of 60, 75 or 100 mg/m² once every three weeks.

In a particularly preferred aspect of this embodiment, the invention provides a method of treating prostate cancer in a patient, such as a human, by administering to the patient Compound 1 in an amount of 25 to 50, preferably 25, 37.5 or 50 mg daily, on a 4/2 or 2/1 dosing schedule, and docetaxel in an infusion of 60, 75 or 100 mg/m² once every three weeks.

In a particularly preferred aspect of this embodiment, the invention provides a method of treating prostate cancer in a patient, such as a human, by administering to the patient Compound 1 in an amount of 25 to 50, preferably 25, 37.5 or 50 mg daily, on a continuous dosing schedule, and docetaxel in an infusion of 60, 75 or 100 mg/m² once every three weeks.

(5) Gemzar™/gemcitabine: In another embodiment, the invention provides combination therapies of Compound 1 and gemcitabine, a compound available from Eli Lilly and Company as a lyophilate for injection in single-use vials containing 200 mg or 1 g (free base equivalent) gemcitabine HCl, under the tradename Gemzar™. Preferably, the combination is used to treat a patient, preferably a human, suffering from cancer. Thus, in a particular aspect of this embodiment, the invention provides a method of treating cancer by administering to a patient Compound 1 and gemcitabine in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Gemcitabine, diluted for infusion as directed by the manufacturer can be administered in a dosage of from 750 to 1250 mg/m², preferably 750, 1000 or 1250 mg/m², as a 30-minute bolus infusion once weekly for 2, 3 or 4 weeks, followed by a one-week rest period. Compound 1 and gemcitabine can be administered at the same time, or sequentially, without regard to order. Compound 1 can be administered continuously (i.e., daily for the duration of the treatment), or more preferably, in an intermittent dosing regimen, and gemcitabine can be administered once weekly on an intermittent dosing schedule without regard to the Compound 1 dosing schedule. Alternatively, the dosing regimens can be chosen so that rest periods for Compound 1 and gemcitabine coincide. Both Compound 1 and gemcitabine can be administered in a fed or fasted state. In a particularly preferred embodiment, Compound 1 is administered in an amount of 25 to 50, preferably 25, 37.5 or 50 mg daily, on a 4/2 or 2/1 dosing schedule, and gemcitabine is administered in an infusion of 750 or 1000 or 1250 mg/m² once weekly for 2 or 3 or 4 weeks followed by a one-week gemcitabine rest period. In another particularly preferred embodiment, Compound 1 is administered in an amount of 25 to 50, preferably 25, 37.5 or 50 mg daily, on a continuous dosing schedule, and gemcitabine is administered in an infusion of 750 or 1000 or 1250 mg/m² once weekly for 2 or 3 or 4 weeks followed by a one-week gemcitabine rest period.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating pancreatic cancer in a patient, such as a human, by administering to the patient Compound 1 in an amount of 25 to 50, preferably 25, 37.5 or 50 mg daily, on a 4/2 or 2/1 dosing schedule, and gemcitabine in an infusion of 750 or 1000 or 1250 mg/m² once weekly for 2 or 3 or 4 weeks followed by a one-week gemcitabine rest period.

In another particularly preferred aspect of this embodiment, the invention provides a method of treating pancreatic cancer in a patient, such as a human, by administering to the patient Compound 1 in an amount of 25 to 50, preferably 25, 37.5 or 50 mg daily, on a continuous dosing schedule, and gemcitabine in an infusion of 750 or 1000 or 1250 mg/m² once weekly for 2 or 3 or 4 weeks followed by a one-week gemcitabine rest period.

In another particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a 4/2 or 2/1 dosing schedule, and gemcitabine in an infusion of 750 or 1000 or 1250 mg/m² once weekly for 2 or 3 or 4 weeks followed by a one-week gemcitabine rest period.

In another particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a continuous dosing schedule, and gemcitabine in an infusion of 750 or 1000 or 1250 mg/m² once weekly for 2 or 3 or 4 weeks followed by a one-week gemcitabine rest period.

In another particularly preferred aspect of this embodiment, the invention provides a method of treating bladder cancer in a patient, such as a human, by administering to the patient Compound 1 in an amount of 25 to 50, preferably 25, 37.5 or 50 mg daily, on a 4/2 or 2/1 dosing schedule, and gemcitabine in an infusion of 750 or 1000 or 1250 mg/m² once weekly for 2 or 3 or 4 weeks followed by a one-week gemcitabine rest period.

In another particularly preferred aspect of this embodiment, the invention provides a method of treating bladder cancer in a patient, such as a human, by administering to the patient Compound 1 in an amount of 25 to 50, preferably 25, 37.5 or 50 mg daily, on a continuous dosing schedule, and gemcitabine in an infusion of 750 or 1000 or 1250 mg/m² once weekly for 2 or 3 or 4 weeks followed by a one-week gemcitabine rest period.

(6) Xeloda™/capecitabine: In another embodiment, the invention provides combination therapies of Compound 1 and, capecitabine, a compound available from Roche as a 150 or 500 mg tablet under the tradename Xeloda™. Preferably, the combination is used to treat a patient, preferably a human, suffering from cancer. Thus, in a particular aspect of this embodiment, the invention provides a method of treating cancer by administering to a patient Compound 1 and capecitabine in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Capecitabine can be administered in a dosage of from 675 to 1250 mg/m² twice daily, preferably 825 or 1000 or 1250 mg/m² twice daily orally. Compound 1 and one of the two daily doses of capecitabine can be administered at the same time, or sequentially, without regard to order. Compound 1 and capecitabine can be administered continuously (i.e., daily for the duration of the treatment). More preferably, Compound 1 is administered in an intermittent dosing regimen such as a 4/2 or 2/1 dosing regimen, and capecitabine is administered in an intermittent dosing regimen such as a 2/1 dosing regimen. If both Compound 1 and capecitabine are administered in 2/1 dosing regimens, preferably the regimens are synchronized so that the treatment periods of the two compounds coincide and the rest periods of the two compounds coincide. Compound 1 can be administered in a fed or fasted state, but capecitabine is preferably administered in a fed state, preferably within 30 minutes after a meal. In a particularly preferred embodiment, Compound 1 is administered in an amount of 25 to 50, preferably 25, 37.5 or 50 mg daily, on a 4/2 or 2/1 dosing schedule, and capecitabine is administered twice daily in an amount of 825 or 1000 or 1250 mg/m² on a 2/1 dosing schedule. In another particularly preferred embodiment, Compound 1 is administered in an amount of 25 to 50, preferably 25, 37.5 or 50 mg daily, on a continuous dosing schedule, and capecitabine is administered twice daily in an amount of 825 or 1000 or 1250 mg/m² on a 2/1 dosing schedule.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating breast cancer in a patient, such as a human, by administering to the patient Compound 1 in an amount of 25 to 50, preferably 25, 37.5 or 50 mg daily, on a 4/2 or 2/1 dosing schedule, and capecitabine in an amount of 825 or 1000 or 1250 mg/m² twice daily on a 2/1 dosing schedule.

In another particularly preferred aspect of this embodiment, the invention provides a method of treating breast cancer in a patient, such as a human, by administering to the patient Compound 1 in an amount of 25 to 50, preferably 25, 37.5 or 50 mg daily, on a continuous dosing schedule, and capecitabine in an amount of 825 or 1000 or 1250 mg/m² twice daily on a 2/1 dosing schedule.

In another particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a 4/2 or 2/1 dosing schedule, and capecitabine in an amount of 825 or 1000 or 1250 mg/m² twice daily on a 2/1 dosing schedule.

In another particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a continuous dosing schedule, and capecitabine in an amount of 825 or 1000 or 1250 mg/m² twice daily on a 2/1 dosing schedule.

(7) Folfox: In another embodiment, the invention provides combination therapies of Compound 1 and FOLFOX, a combination of oxaliplatin, 5-fluorouracil (“5-FU”) and leucovorin described in R. M. Goldberg et al., “N9741: FOLFOX (oxaliplatin(Oxal)/5-fluorouracil (5-FU)/leucovorin (LV) or reduced dose R-IFL (CPT-11+5-FU/LV) in advanced colorectal cancer (CRC): Final efficacy data from an intergroup study”, Journal of Clinical Oncology, 2004 ASCO Annual Meeting Proceedings (Post-Meeting Edition), Vol. 22, No 14S (July 15 Supplement), 2004: 362, the disclosure of which is incorporated herein by reference in its entirety. Oxaliplatin is available as a lyophilate for reconstitution in 50 and 100 mg vials from Sanofi-Synthelabo under the tradename Eloxatin™. 5-Fluorouracil is available as a solution for injection in 500 mg vials (50 mg/mL, 10 mL) from Pfizer, Inc., under the tradename Adrucil™. Leucovorin, also known as LV, calcium leucovorin, folinic acid, calcium folinate or citrovorum factor, is available from several sources, including Wyeth Pharmaceuticals (Lederle Leucovorin™ Calcium). Preferably, the combination is used to treat a patient, preferably a human, suffering from cancer. Thus, in a particular aspect of this embodiment, the invention provides a method of treating cancer by administering to a patient Compound 1 and FOLFOX in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Compound 1 can be administered continuously (i.e., daily for the duration of the treatment), or more preferably, in an intermittent dosing regimen, in a fed or fasted state. FOLFOX can be administered according to the standard FOLFOX4 dosing schedule well known in the art. In particular, FOLFOX4 can be administered on days 1 and 2 of each two week cycle, as follows. On Day 1, 85 mg/m² oxaliplatin and 200 mg/m² leucovorin are administered simultaneously in a two-hour infusion, followed by an intravenous bolus of 400 mg/m² 5-FU and 600 Mg/m² of 5-FU in a 22-hour infusion. On Day 2, 200 Mg/m² leucovorin is administered in a two-hour infusion, followed by an intravenous bolus of 400 mg/m² 5-FU and 600 mg/m² of 5-FU in a 22-hour infusion. Days 3-14 of the FOLFOX regime are a FOLFOX rest period. In a particularly preferred embodiment, Compound 1 is administered in an amount of 25 to 50, preferably 25, 37.5 or 50 mg daily, on a 4/2 or 2/2 dosing schedule, and FOLFOX4 is administered as described herein. In another particularly preferred embodiment, Compound 1 is administered in an amount of 25 to 50, preferably 26, 37.5 or 50 mg daily, on a continuous dosing schedule, and FOLFOX4 is administered as described herein.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a 4/2 or 2/2 dosing schedule, and FOLFOX on a standard FOLFOX4 dosing regimen as described above.

In another particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a continuous dosing schedule, and FOLFOX on a standard FOLFOX4 dosing regimen as described above.

(8) Folfiri: In another embodiment, the invention provides combination therapies of Compound 1 and FOLFIRI, a combination of irinotecan, 5-fluorouracil (“5-FU”) and leucovorin. Irinotecan, also known as CPT-11, is available from Pfizer, Inc. as a solution for dilution and injection in 2 and 5 mL vials (40 and 100 mg irinotecan hydrochloride, respectively) under the tradename Camptosar™ (irinotecan hydrochloride injection). 5-Fluorouracil is available as a solution for injection in 500 mg vials (50 mg/mL, 10 mL) from Pfizer, Inc., under the tradename Adrucil™. Leucovorin, also known as LV, calcium leucovorin, folinic acid, calcium folinate or citrovorum factor, is available from several sources, including Wyeth Pharmaceuticals (Lederle Leucovorin™ Calcium). Preferably, the combination is used to treat a patient, preferably a human, suffering from cancer. Thus, in a particular aspect of this embodiment, the invention provides a method of treating cancer by administering to a patient Compound 1 and FOLFIRI in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Compound 1 can be administered continuously (i.e., daily for the duration of the treatment), or more preferably, in an intermittent dosing regimen, in a fed or fasted state. FOLFIRI can be administered according to the standard dosing schedule well known in the art. In particular, FOLFIRI can be administered every two weeks, as follows. On Day 1 of each two week cycle, 180 mg/m² irinotecan is administered in a 90-minute infusion, and 200 mg/m² leucovorin is administered concurrently with the irinotecan in a two-hour infusion, followed by an intravenous bolus of 400-500 mg/m² 5-FU. Then, 2400-3000 mg/m² of 5-FU is administered in a 46-hour infusion. Days 3-14 of the FOLFIRI regime are a FOLFIRI rest period. In a particularly preferred embodiment, Compound 1 is administered in an amount of 25 to 50, preferably 25, 37.5 or 50 mg daily, on a 4/2 or 2/2 dosing schedule, and FOLFIRI is administered as described herein. In another particularly preferred embodiment, Compound 1 is administered in an amount of 25 to 50, preferably 25, 37.5 or 50 mg daily, on a continuous dosing schedule, and FOLFIRI is administered as described herein.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a 4/2 or 2/2 dosing schedule, and FOLFIRI on a standard FOLFIRI dosing regimen as described above.

In another particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a continuous dosing schedule, and FOLFIRI on a standard FOLFIRI dosing regimen as described above.

(9) Gleevec™/imatinib: In another embodiment, the invention provides combination therapies of Compound 1 and, imatinib, a compound available from Novartis as a tablet containing imatinib mesylate in 100 or 400 mg free base equivalent under the tradename Gleevec™. Preferably, the combination is used to treat a patient, preferably a human, suffering from cancer. Thus, in a particular aspect of this embodiment, the invention provides a method of treating cancer by administering to a patient Compound 1 and imatinib in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Imatinib can be administered in a dosage of 400 or 600 mg once daily. Compound 1 and imatinib can be administered at the same time, or sequentially, without regard to order. Compound 1 and capecitabine can be administered continuously (i.e., daily for the duration of the treatment). More preferably, Compound 1 is administered in an intermittent dosing regimen such as a 4/2 dosing regimen, and imatinib is administered in a continuous (once daily) dosing regimen. Compound 1 can be administered in a fed or fasted state, but imatinib is preferably administered with food and water to minimize potential adverse gastrointestinal effects. In a particularly preferred embodiment, Compound 1 is administered in an amount of 25 to 50, preferably 25, 37.5 or 50 mg daily, on a 4/2 dosing schedule, and imatinib is administered once daily in an amount of 400 to 600 mg on a continuous dosing schedule. In another particularly preferred embodiment, Compound 1 is administered in an amount of 25 to 50, preferably 25, 37.5 or 50 mg daily, on a continuous dosing schedule, and imatinib is administered once daily in an amount of 400 to 600 mg on a continuous dosing schedule.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating gastrointestinal stromal tumors (GIST) in a patient, such as a human, by administering to the patient Compound 1 in an amount of 25 to 50, preferably 25, 37.5 or 50 mg daily, on a 4/2 dosing schedule, and imatinib once daily in an amount of 400 to 600 mg on a continuous dosing schedule.

In another particularly preferred aspect of this embodiment, the invention provides a method of treating gastrointestinal stromal tumors (GIST) in a patient, such as a human, by administering to the patient Compound 1 in an amount of 25 to 50, preferably 25, 37.5 or 50 mg daily, on a continuous dosing schedule, and imatinib once daily in an amount of 400 to 600 mg on a continuous dosing schedule.

(10) Herceptin™/trastuzumab: In another embodiment, the invention provides combination therapies of Compound 1 and trastuzumab, a monoclonal antibody available from Genentech as a lyophilate for injection in single-use vials containing 440 mg trastuzumab, under the tradename Herceptin™. Preferably, the combination is used to treat a patient, preferably a human, suffering from cancer. Thus, in a particular aspect of this embodiment, the invention provides a method of treating cancer by administering to a patient Compound 1 and trastuzumab in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Trastuzumab, diluted for infusion as directed by the manufacturer can be administered in an initial loading dose of 4 mg/kg as a 90-minute infusion, followed by once-weekly maintenance doses of 2 mg/kg as a 30-minute infusion for the duration of the treatment. Compound 1 and trastuzumab can be administered without regard to order. Compound 1 can be administered continuously (i.e., daily for the duration of the treatment), or more preferably, in an intermittent dosing regimen, and trastuzumab can be administered once weekly without regard to the Compound 1 dosing schedule. Both Compound 1 and trastuzumab can be administered in a fed or fasted state. In a particularly preferred embodiment, Compound 1 is administered in an amount of 25 to 50, preferably 25, 37.5 or 50 mg daily, on a 4/2 dosing schedule, and trastuzumab is administered once weekly as described above. In another particularly preferred embodiment, Compound 1 is administered in an amount of 25 to 50, preferably 25, 37.5 or 50 mg daily, on a continuous dosing schedule, and trastuzumab is administered once weekly as described above.

In a particular aspect of this embodiment, the cancer is breast cancer, particularly HER2 positive breast cancer. As used herein, the term “HER2 positive” means characterized by HER2 protein overexpression, and such overexpression can be determined by methods well known in the art, such as by immunohistochemistry (IHC) or fluorescence in situ hybridization (FISH). A HER2 IHC assay is available commercially from DakoCytomation, Carpinteria, Calif., USA, under the tradename HercepTest™. A HER2 FISH assay is available commercially from Vysis, Inc., Downers Grove, Ill., USA, under the tradename PathVysion™. HER2 assays are described in the literature in, for example, M. F. Press et al., “Her-2/neu expression in noe-negative breast cancer: direct tissue quantitation by computerized image analysis and association of overexpression with increased risk of recurrent disease,” Cancer Res. 1993, 53, 4960-4970, the disclosure of which is incorporated herein by reference in its entirety.

In a particularly preferred aspect of this embodiment, the invention provides a method of treating HER2 positive breast cancer in a patient, such as a human, by administering to the patient Compound 1 in an amount of 25 to 50, preferably 25, 37.5 or 50 mg daily, on a 4/2 dosing schedule, and trastuzumab in an initial loading dose of 4 mg/kg, followed by once-weekly doses of 2 mg/kg.

In another particularly preferred aspect of this embodiment, the invention provides a method of treating HER2 positive breast cancer in a patient, such as a human, by administering to the patient Compound 1 in an amount of 25 to 50, preferably 25, 37.5 or 50 mg daily, on a continuous dosing schedule, and trastuzumab in an initial loading dose of 4 mg/kg, followed by once-weekly doses of 2 mg/kg.

(11) Alimta™/pemetrexed: In another embodiment, the invention provides combination therapies of Compound 1 and pemetrexed, a compound available from Eli Lilly and Company as a lyophilate for injection in single-use vials containing 500 mg pemetrexed, under the tradename Alimta™ (pemetrexed for injection). Preferably, the combination is used to treat a patient, preferably a human, suffering from cancer. Thus, in a particular aspect of this embodiment, the invention provides a method of treating cancer by administering to a patient Compound 1 and pemetrexed in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Pemetrexed, diluted for infusion as directed by the manufacturer can be administered in a dosage of from 250 to 500 mg/m², preferably 250, 375 or 500 mg/m², more preferably 500 mg/m², as an infusion over 30 minutes, once every 3 weeks. Compound 1 and pemetrexed can be administered without regard to order. Compound 1 can be administered continuously (i.e., daily for the duration of the treatment), or more preferably, in an intermittent dosing regimen, such as 4/2 or 2/1, and pemetrexed can be administered once every 3 weeks without regard to the Compound 1 dosing schedule. Alternatively, the dosing regimens can be chosen so that the pemetrexed treatment occurs on the first day of each Compound 1 2/1 treatment cycle. Both Compound 1 and pemetrexed can be administered in a fed or fasted state. In a particularly preferred embodiment, Compound 1 is administered in an amount of 25 to 50, preferably 25, 37.5 or 50 mg daily, on a 4/2 or 2/1 dosing schedule, and pemetrexed is administered in an infusion of 250 to 500 mg/m², preferably 250 or 375 or 500 mg/m² once every three weeks. In another particularly preferred embodiment, Compound 1 is administered in an amount of 25 to 50, preferably 25, 37.5 or 50 mg daily, on a continuous dosing schedule, and pemetrexed is administered in an infusion of 250 to 500 mg/m², preferably 250 or 375 or 500 mg/m² once every three weeks.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma Of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a 4/2 or 2/1 dosing schedule, and pemetrexed in an infusion of 250 to 500 mg/m², preferably 250 or 375 or 500 mg/m² once every three weeks.

In another particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a continuous dosing schedule, and pemetrexed in an infusion of 250 to 500 mg/m², preferably 250 or 375 or 500 mg/m² once every three weeks.

(12) Aromatase inhibitors: In another embodiment, the invention is directed to combination treatments using Compound 1 and an aromatase inhibitor. Compound 1 can be administered in the dosage amounts and schedules described herein. Suitable aromatase inhibitors include steroidal aromatase inhibitors, such as exemestane (Aromasin™, Pfizer, Inc.), and non-steroidal aromatase inhibitors, such as anastrozole (Arimidex™, AstraZeneca) and letrozole (Femara™, Novartis).

(12a) Aromasin™/exemestane: In one aspect of this embodiment, the aromatase inhibitor is exemestane, available from Pfizer, Inc. as a 25 mg tablet under the tradename Aromasin™. Preferably, the combination is used to treat a patient, preferably a human, suffering from breast cancer. Thus, in a particular aspect of this embodiment, the invention provides a method of treating breast cancer by administering to a patient Compound 1 and exemestane in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Exemestane can be administered in a dosage of 25 mg once daily, preferably orally. Compound 1 and exemestane can be administered at the same time, or sequentially, without regard to order. Compound 1 and exemestane can be administered continuously (i.e., daily for the duration of the treatment). More preferably, Compound 1 is administered in an intermittent dosing regimen, preferably a 4/2 dosing regimen, and exemestane is administered continuously (once daily). Compound 1 can be administered in a fed or fasted state, but exemestane is administered with food (after a meal). In a particularly preferred embodiment, Compound 1 is administered in an amount of 25 to 50, preferably 25, 37.5 or 50 mg daily, on a 4/2 dosing schedule, and exemestane is administered once daily in an amount of 25 mg. In another particularly preferred embodiment, Compound 1 is administered in an amount of 25 to 50, preferably 25, 37.5 or 50 mg daily, on a continuous dosing schedule, and exemestane is administered once daily in an amount of 25 mg

(12b) Arimidex™/anastrozole: In another aspect of this embodiment, the aromatase inhibitor is anastrozole, available from AstraZeneca as a 1 mg tablet under the tradename Arimidex™. Preferably, the combination is used to treat a patient, preferably a human, suffering from breast cancer. Thus, in a particular aspect of this embodiment, the invention provides a method of treating breast cancer by administering to a patient Compound 1 and anastrozole in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Anastrozole can be administered in a dosage of 1 mg once daily, preferably orally. Compound 1 and anastrozole can be administered at the same time, or sequentially, without regard to order. Compound 1 and anastrozole can be administered continuously (i.e., daily for the duration of the treatment). More preferably, Compound 1 is administered in an intermittent dosing regimen, preferably a 4/2 dosing regimen, and anastrozole is administered continuously (once daily). Both Compound 1 and anastrozole can be administered in a fed or fasted state. In a particularly preferred embodiment, Compound 1 is administered in an amount of 25 to 50, preferably 25, 37.5 or 50 mg daily, on a 4/2 dosing schedule, and anastrozole is administered once daily in an amount of 1 mg. In another particularly preferred embodiment, Compound 1 is administered in an amount of 25 to 50, preferably 25, 37.5 or 50 mg daily, on a continuous dosing schedule, and anastrozole is administered once daily in an amount of 1 mg.

(12c) Femara™/letrozole: In another aspect of this embodiment, the aromatase inhibitor is letrozole, available from Novartis as a 2.5 mg tablet under the tradename Femara™. Preferably, the combination is used to treat a patient, preferably a human, suffering from breast cancer. Thus, in a particular aspect of this embodiment, the invention provides a method of treating breast cancer by administering to a patient Compound 1 and letrozole in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Letrozole can be administered in a dosage of 2.5 mg once daily, preferably orally. Compound 1 and letrozole can be administered at the same time, or sequentially, without regard to order. Compound 1 and letrozole can be administered continuously (i.e., daily for the duration of the treatment). More preferably, Compound 1 is administered in an intermittent dosing regimen, preferably a 4/2 dosing regimen, and letrozole is administered continuously (once daily). Both Compound 1 and letrozole can be administered in a fed or fasted state. In a particularly preferred embodiment, Compound 1 is administered in an amount of 25 to 50, preferably 25, 37.5 or 50 mg daily, on a 4/2 dosing schedule, and letrozole is administered once daily in an amount of 2.5 mg. In another particularly preferred embodiment, Compound 1 is administered in an amount of 25 to 50, preferably 25, 37.5 or 50 mg daily, on a continuous dosing schedule, and letrozole is administered once daily in an amount of 2.5 mg.

(13) Temodar™/temozolomide: In another embodiment, the invention provides combination therapies of Compound 1 and, temozolomide, a compound available from Schering Corporation as a 5, 20, 100 or 250 mg capsule under the tradename Temodar™. Preferably, the combination is used to treat a patient, preferably a human, suffering from cancer. Thus, in a particular aspect of this embodiment, the invention provides a method of treating cancer by administering to a patient Compound 1 and temozolomide in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Temozolomide can be administered in a dosage of from 75 to 200 mg/m² once daily, preferably 75, 150 or 200 mg/m² once daily orally. Compound 1 and temozolomide can be administered at the same time, or sequentially, without regard to order. Compound 1 can be administered continuously (i.e., daily for the duration of the treatment). More preferably, Compound 1 is administered in an intermittent dosing regimen such as a 4/2, 3/1 or 2/2 dosing regimen. Temozolomide is administered in an intermittent dosing regimen. In general, temozolomide is administered at a dose of 150 or 200 mg/m² on the first 5 days of a 4-week treatment cycle, with days 6 through 28 of each cycle being a temozolomide rest period. If desired, particularly for newly diagnosed high grade gliomas, this temozolomide regimen can be preceded by a 6-week regimen of temozolomide at a dose of 75 mg/m² daily. If Compound 1 is administered in a 3/1 or 2/2 schedule, preferably the schedules are synchronized so that the 5-day temozolomide treatment period of each 4-week temozolomide cycle coincides with the first 5-days of Compound 1 treatment in the 3/1 or 2/2 treatment cycle. Compound 1 can be administered in a fed or fasted state, but temozolomide is preferably administered on an empty stomach. In a particularly preferred embodiment, Compound 1 is administered in an amount of 25 to 50, preferably 25, 37.5 or 50 mg daily, on a 4/2 or 3/1 or 2/1 dosing schedule, and temozolomide is administered once daily in an amount of 150 to 200 mg/m² on the first 5 days only of a 4-week dosing schedule. In another particularly preferred embodiment, Compound 1 is administered in an amount of 25 to 50, preferably 25, 37.5 or 50 mg daily, on a continuous dosing schedule, and temozolomide is administered once daily in an amount of 150 to 200 mg/m² on the first 5 days only of a 4-week dosing schedule.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating brain tumors in a patient, such as a human, by administering to the patient Compound 1 in an amount of 25 to 50, preferably 25, 37.5 or 50 mg daily, on a 4/2 or 3/1 or 2/1 dosing schedule, and temozolomide once daily in an amount of 150 to 200 mg/m² on the first 5 days only of a 4-week dosing schedule. In a more particularly preferred aspect of this embodiment, the brain tumor is anaplastic astrocytoma. In another particularly preferred aspect of this embodiment, the brain tumor is glioblastoma.

In another particularly preferred aspect of this embodiment, the invention provides a method of treating brain tumors in a patient, such as a human, by administering to the patient Compound 1 in an amount of 25 to 50, preferably 25, 37.5 or 50 mg daily, on a continuous dosing schedule, and temozolomide once daily in an amount of 150 to 200 mg/m² on the first 5 days only of a 4-week dosing schedule. In a more particularly preferred aspect of this embodiment, the brain tumor is anaplastic astrocytoma. In another particularly preferred aspect of this embodiment, the brain tumor is glioblastoma.

In another particularly preferred aspect of this embodiment, the invention provides a method of treating melanoma in a patient, such as a human, by administering to the patient Compound 1 in an amount of 25 to 50, preferably 25, 37.5 or 50 mg daily, on a 4/2 or 3/1 or 2/1 dosing schedule, and temozolomide once daily in an amount of 150 to 200 mg/m² on the first 5 days only of a 4-week dosing schedule.

In another particularly preferred aspect of this embodiment, the invention provides a method of treating melanoma in a patient, such as a human, by administering to the patient Compound 1 in an amount of 25 to 50, preferably 25, 37.5 or 50 mg daily, on a continuous dosing schedule, and temozolomide once daily in an amount of 150 to 200 mg/m² on the first 5 days only of a 4-week dosing schedule.

(14) DTIC™-Dome/dacarbazine: In another embodiment, the invention provides combination therapies of Compound 1 and, dacarbazine, a compound available from Bayer as a lyophilate for injection in 100 or 200 mg vials under the tradename DTIC™-Dome. Preferably, the combination is used to treat a patient, preferably a human, suffering from cancer. Thus, in a particular aspect of this embodiment, the invention provides a method of treating cancer by administering to a patient Compound 1 and dacarbazine in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Dacarbazine can be administered intravenously in a dosage of from 2 to 4.5 mg/kg/day or alternatively 250 mg/m²/day. Compound 1 and dacarbazine can be administered without regard to order. Compound 1 can be administered continuously (i.e., daily for the duration of the treatment). More preferably, Compound 1 is administered in an intermittent dosing regimen such as a 4/2, 3/1, 2/2 or 2/1 dosing regimen. Dacarbazine is administered in an intermittent dosing regimen. In one regimen, dacarbazine is administered intravenously at a dose of 2 to 4.5 mg/kg/day on the first 10 days of a 4-week treatment cycle, with days 11 through 28 of each cycle being a dacarbazine rest period. In another regimen, dacarbazine is administered intravenously at a dose of 250 mg/m²/day on the first 5 days of a 3-week treatment cycle, with days 6 through 21 of each cycle being a dacarbazine rest period. If Compound 1 is administered in a 3/1 or 2/2 schedule and dacarbazine is administered in a 4-week cycle as described above, preferably the schedules are synchronized so that the 10-day dacarbazine treatment period of each 4-week dacarbazine cycle coincides with the first 10 days of Compound 1 treatment in the 3/1 or 2/2 treatment cycle. If Compound 1 is administered in a 2/1 schedule and dacarbazine is administered in a 3-week cycle as described above, preferably the schedules are synchronized so that the 5-day dacarbazine treatment period of each 3-week dacarbazine cycle coincides with the first 5 days of Compound 1 treatment in the 2/1 treatment cycle. Both Compound 1 and dacarbazine can be administered in a fed or fasted state.

In a particularly preferred embodiment, Compound 1 is administered in an amount of 25 to 50, preferably 25, 37.5 or 50 mg daily, on a 4/2 dosing schedule, and dacarbazine is administered in a 4-week or 3-week cycle as described above.

In another particularly preferred embodiment, Compound 1 is administered in an amount of 25 to 50, preferably 25, 37.5 or 50 mg daily, on a 3/1 or 2/2 dosing schedule, and dacarbazine is administered intravenously at a dose of 2 to 4.5 mg/kg/day on the first 10 days only of a 4-week treatment cycle.

In another particularly preferred embodiment, Compound 1 is administered in an amount of 25 to 50, preferably 25, 37.5 or 50 mg daily, on a 2/1 dosing schedule, and dacarbazine is administered intravenously at a dose of 250 mg/m²/day on the first 5 days only of a 3-week treatment cycle.

In a particularly preferred embodiment, Compound 1 is administered in an amount of 25 to 50, preferably 25, 37.5 or 50 mg daily, on a continuous dosing schedule, and dacarbazine is administered in a 4-week or 3-week cycle as described above.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating melanoma in a patient, such as a human, by administering to the patient Compound 1 in an amount of 25 to 50, preferably 25, 37.5 or 50 mg daily, on a 4/2 dosing schedule, and dacarbazine in a 4-week or 3-week cycle as described above.

In another particularly preferred aspect of this embodiment, the invention provides a method of treating melanoma in a patient, such as a human, by administering to the patient Compound 1 in an amount of 25 to 50, preferably 25, 37.5 or 50 mg daily, on a 3/1 or 2/2 dosing schedule, and dacarbazine intravenously at a dose of 2 to 4.5 mg/kg/day on the first 10 days only of a 4-week treatment cycle.

In another particularly preferred aspect of this embodiment, the invention provides a method of treating melanoma in a patient, such as a human, by administering to the patient Compound 1 in an amount of 25 to 50, preferably 25, 37.5 or 50 mg daily, on a 2/1 dosing schedule, and dacarbazine intravenously at a dose of 250 mg/m²/day on the first 5 days only of a 3-week treatment cycle.

In another particularly preferred aspect of this embodiment, the invention provides a method of treating melanoma in a patient, such as a human, by administering to the patient Compound 1 in an amount of 25 to 50, preferably 25, 37.5 or 50 mg daily, on a continuous dosing schedule, and dacarbazine in a 4-week or 3-week cycle as described above.

(15) Avastin™/bevacizumab: In another embodiment, the invention provides combination therapies of Compound 1 and bevacizumab, a monoclonal antibody available from Genentech as a lyophilate for injection in single-use vials containing 100 or 400 mg bevacizumab, under the tradename Avastin™. Preferably, the combination is used to treat a patient, preferably a human, suffering from cancer. Thus, in a particular aspect of this embodiment, the invention provides a method of treating cancer by administering to a patient Compound 1 and bevacizumab in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Bevacizumab, diluted for infusion as directed by the manufacturer can be administered in a dosage of from 3 to 10 mg/kg, preferably 5 mg/kg, as an infusion over 30, 60 or 90 minutes, once every 2 weeks. Compound 1 and bevacizumab can be administered without regard to order. Compound 1 can be administered continuously (i.e., daily for the duration of the treatment), or more preferably, in an intermittent dosing regimen, such as 4/2, and bevacizumab can be administered once every 2 weeks without regard to the Compound 1 dosing schedule. Preferably, Compound 1 is administered on a 4/2 dosing schedule, and the dosing regimens are synchronized so that the bevacizumab treatment occurs on days 1, 15 and 29 of the Compound 1 4/2 treatment cycle. Both Compound 1 and bevacizumab can be administered in a fed or fasted state. In a particularly preferred embodiment, Compound 1 is administered in an amount of 25 to 50, preferably 25, 37.5 or 50 mg daily, on a 4/2 dosing schedule, and bevacizumab is administered in an infusion of 3 to 10 mg/kg, preferably 3, 5 or 10 mg/kg, once every two weeks. In another particularly preferred embodiment, Compound 1 is administered in an amount of 25 to 50, preferably 25, 37.5 or 50 mg daily, on a continuous dosing schedule, and bevacizumab is administered in an infusion of 3 to 10 mg/kg, preferably 3, 5 or 10 mg/kg, once every two weeks.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating renal cell carcinoma in a patient, such as a human, by administering to the patient Compound 1 in an amount of 25 to 50, preferably 25, 37.5 or 50 mg daily, on a 4/2 dosing schedule, and bevacizumab in an infusion of 3 to 10 mg/kg, preferably 3, 5 or 10 mg/kg, once every two weeks.

In another particularly preferred aspect of this embodiment, the invention provides a method of treating renal cell carcinoma in a patient, such as a human, by administering to the patient Compound 1 in an amount of 25 to 50, preferably 25, 37.5 or 50 mg daily, on a continuous dosing schedule, and bevacizumab in an infusion of 3 to 10 mg/kg, preferably 3, 5 or 10 mg/kg, once every two weeks.

In another particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a 4/2 dosing schedule, and bevacizumab in an infusion of 3 to 10 mg/kg, preferably 3, 5 or 10 mg/kg, once every two weeks.

In another particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a continuous dosing schedule, and bevacizumab in an infusion of 3 to 10 mg/kg, preferably 3, 5 or 10 mg/kg, once every two weeks.

In another particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a 4/2 dosing schedule, and bevacizumab in an infusion of 3 to 10 mg/kg, preferably 3, 5 or 10 mg/kg once every two weeks.

In another particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a continuous dosing schedule, and bevacizumab in an infusion of 3 to 10 mg/kg, preferably 3, 5 or 10 mg/kg, once every two weeks.

In another particularly preferred aspect of this embodiment, the invention provides a method of treating prostate cancer in a patient, such as a human, by administering to the patient Compound 1 in an amount of 25 to 50, preferably 25, 37.5 or 50 mg daily, on a 4/2 dosing schedule, and bevacizumab in an infusion of 3 to 10 mg/kg, preferably 3, 5 or 10 mg/kg, once every two weeks.

In another particularly preferred aspect of this embodiment, the invention provides a method of treating prostate cancer in a patient, such as a human, by administering to the patient Compound 1 in an amount of 25 to 50, preferably 25, 37.5 or 50 mg daily, on a continuous dosing schedule, and bevacizumab in an infusion of 3 to 10 mg/kg, preferably 3, 5 or 10 mg/kg, once every two weeks.

In another particularly preferred aspect of this embodiment, the invention provides a method of treating breast cancer in a patient, such as a human, by administering to the patient Compound 1 in an amount of 25 to 50, preferably 25, 37.5 or 50 mg daily, on a 4/2 dosing schedule, and bevacizumab in an infusion of 3 to 10 mg/kg, preferably 3, 5 or 10 mg/kg, once every two weeks.

In another particularly preferred aspect of this embodiment, the invention provides a method of treating breast cancer in a patient, such as a human, by administering to the patient Compound 1 in an amount of 25 to 50, preferably 25, 37.5 or 50 mg daily, on a continuous dosing schedule, and bevacizumab in an infusion of 3 to 10 mg/kg, preferably 3, 5 or 10 mg/kg, once every two weeks.

(16) Anthracyclines: In another embodiment, the invention provides combination therapies of Compound 1 and an anthracycline. Compound 1 can be administered in the dosage amounts and schedules described herein. Suitable anthracyclines include daunorubicin (Cerubidine™, Bedford Laboratories), idarubicin (Idamycin™, Pharmacia & Upjohn Co.), doxorubicin (Adriamycin™, Pharmacia & Upjohn Co.) and epirubicin (Ellence™, Pharmacia & Upjohn Co.).

(16a) Adriamycin™/doxorubicin: In one aspect of this embodiment, the anthracycline is doxorubicin, a compound available from Pharmacia & Upjohn Co. (Pfizer, Inc.) as a lyophilate for injection in single-use vials containing 10, 20 or 50 mg, and in a multiple-use vial containing 150 mg, of doxorubicin hydrochloride, under the tradename Adriamycin RDF™ (doxorubicin hydrochloride for injection). Doxorubicin is also available from Pharmacia & Upjohn Co. (Pfizer, Inc.) as a sterile, parenteral, isotonic solution for IV use in 5 mL (10 mg), 10 mL (20 mg), 25 mL (50 mg) and 37.5 mL (75 mg) single-dose vials and a 100 mL (200 mg) multidose vial, under the tradename Adriamycin PFS™ (doxorubicin hydrochloride for injection). Preferably, the combination is used to treat a patient, preferably a human, suffering from cancer. Thus, in a particular aspect of this embodiment, the invention provides a method of treating cancer by administering to a patient Compound 1 and doxorubicin in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Doxorubicin can be administered in a dosage of from 40 to 75 mg/m², preferably 40, 60 or 75 mg/m², as a single intravenous injection, once every 3 or 4 weeks. Compound 1 and doxorubicin can be administered without regard to order. Compound 1 can be administered continuously (i.e., daily for the duration of the treatment), or more preferably, in an intermittent dosing regimen, such as 4/2, 3/1 or 2/1, and doxorubicin can be administered once every 3 weeks or once every 4 weeks without regard to the Compound 1 dosing schedule. Preferably, if doxorubicin is administered once every 3 weeks, Compound 1 is administered on a 4/2 dosing schedule, and the dosing regimens are synchronized so that the doxorubicin treatment occurs on days 1 and 22 of each Compound 1 4/2 treatment cycle, or Compound 1 is administered on a 2/1 dosing schedule, and the dosing regimens are synchronized so that the doxorubicin treatment occurs on day 1 of each Compound 1 2/1 treatment cycle, or Compound 1 is administered on a continuous dosing schedule. Preferably, if doxorubicin is administered once every 4 weeks, Compound 1 is administered on a 3/1 dosing schedule, and the dosing regimens are synchronized so that the doxorubicin treatment occurs on day 1 of each Compound 1 3/1 treatment cycle, or Compound 1 is administered on a continuous dosing schedule. Both Compound 1 and doxorubicin can be administered in a fed or fasted state.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating a sarcoma in a patient, such as a human, by administering to the patient Compound 1 in an amount of 25 to 50, preferably 25, 37.5 or 50 mg daily, on a 4/2 dosing schedule, and doxorubicin in an amount of 40 to 75 mg/m², preferably 40, 60 or 75 mg/m², as a single intravenous injection, once every 3 or 4 weeks.

In another particularly preferred aspect of this embodiment, the invention provides a method of treating a sarcoma in a patient, such as a human, by administering to the patient Compound 1 in an amount of 25 to 50, preferably 25, 37.5 or 50 mg daily, on a 3/1 dosing schedule, and doxorubicin in an amount of 40 to 75 mg/m², preferably 40, 60 or 75 mg/m², as a single intravenous injection, once every 3 or 4 weeks.

In another particularly preferred aspect of this embodiment, the invention provides a method of treating a sarcoma in a patient, such as a human, by administering to the patient Compound 1 in an amount of 25 to 50, preferably 25, 37.5 or 50 mg daily, on a continuous dosing schedule, and doxorubicin in an amount of 40 to 75 mg/m², preferably 40, 60 or 75 mg/m², as a single intravenous injection, once every 3 or 4 weeks.

(16b) Ellence™/epirubicin: In another aspect of this embodiment, the anthracycline is epirubicin, a compound available from Pharmacia & Upjohn Co. (Pfizer, Inc.) as a sterile solution for IV use in vials containing 50 or 200 mg epirubicin hydrochloride under the tradename Ellence™ (epirubicin hydrochloride injection). Preferably, the combination is used to treat a patient, preferably a human, suffering from cancer. Thus, in a particular aspect of this embodiment, the invention provides a method of treating cancer by administering to a patient Compound 1 and epirubicin in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Epirubicin can be administered in a dosage of from 60 to 120 mg/m², preferably 60, 75, 90, 100 or 120 mg/m², preferably by injection into a freely flowing intravenous infusion (0.9% sodium chloride or 5% glucose solution) over a period of 3 to 5 minutes, once every 3 or 4 weeks. Optionally, the total dose of epirubicin in each 3 or 4 week cycle can be divided equally and given on days 1 and 8 of each cycle. Compound 1 and epirubicin can be administered without regard to order. Compound 1 can be administered continuously (i.e., daily for the duration of the treatment), or more preferably, in an intermittent dosing regimen, such as 4/2, 3/1 or 2/1, and epirubicin can be administered once every 3 weeks or once every 4 weeks, or divided and given on days 1 and 8 of each 3 or 4-week cycle, without regard to the Compound 1 dosing schedule. Preferably, if epirubicin is administered once every 3 weeks (or divided and given on days 1 and 8 of the 3-week cycle), Compound 1 is administered on a 4/2 dosing schedule, and the dosing regimens are synchronized so that the epirubicin treatment occurs on days 1 (or divided on days 1 and 8) and 22 (or divided on days 22 and 29) of each Compound 1 4/2 treatment cycle, or Compound 1 is administered on a 2/1 dosing schedule, and the dosing regimens are synchronized so that the epirubicin treatment occurs on day 1 (or divided on days 1 and 8) of each Compound 1 2/1 treatment cycle, or Compound 1 is administered on a continuous dosing schedule. Preferably, if epirubicin is administered once every 4 weeks (or divided and given on days 1 and 8 of the 4-week cycle), Compound 1 is administered on a 3/1 dosing schedule, and the dosing regimens are synchronized so that the epirubicin treatment occurs on day 1 (or divided on days 1 and 8) of each Compound 1 3/1 treatment cycle, or Compound 1 is administered on a continuous dosing schedule. Both Compound 1 and epirubicin can be administered in a fed or fasted state.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating a sarcoma in a patient, such as a human, by administering to the patient Compound 1 in an amount of 25 to 50, preferably 25, 37.5 or 50 mg daily, on a 4/2 dosing schedule, and epirubicin in an amount of 60 to 120 mg/m², preferably 60, 75, 90, 100 or 120 mg/m² every 3 or 4 weeks.

In another particularly preferred aspect of this embodiment, the invention provides a method of treating a sarcoma in a patient, such as a human, by administering to the patient Compound 1 in an amount of 25 to 50, preferably 25, 37.5 or 50 mg daily, on a 3/1 dosing schedule, and epirubicin in an amount of 60 to 120 mg/m², preferably 60, 75, 90, 100 or 120 mg/m² every 3 or 4 weeks.

In another particularly preferred aspect of this embodiment, the invention provides a method of treating a sarcoma in a patient, such as a human, by administering to the patient Compound 1 in an amount of 25 to 50, preferably 25, 37.5 or 50 mg daily, on a continuous dosing schedule, and epirubicin in an amount of 60 to 120 mg/m², preferably 60, 75, 90, 100 or 120 mg/m² every 3 or 4 weeks.

(17) Carboplatin-Paclitaxel: In another embodiment, the invention provides combination therapies of Compound 1, carboplatin, and paclitaxel. Carboplatin is a compound available from Bristol-Meyers Squibb Co. as an aqueous solution in 50, 150, 450 and 600 mg multi-dose vials under the tradename Paraplatin™ (carboplatin aqueous solution). Paclitaxel is a compound available from Mead Johnson as a non-aqueous solution for dilution in 30, 100 and 300 mL multi-dose vials under the tradename Taxol™. Preferably, the combination is used to treat a patient, preferably a human, suffering from cancer. Thus, in a particular aspect of this embodiment, the invention provides a method of treating cancer by administering to a patient Compound 1, carboplatin and paclitaxel in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Paclitaxel, diluted for infusion as directed by the manufacturer, can be administered in a dosage of from 135 to 175 mg/m² as an infusion over 3 hours, once every 3 weeks.

Carboplatin dosages are determined as a function of a target area under the concentration versus time curve (AUC in mg/ml/min) and the patient's glomerular filtration rate (GFR in mL/min), a measure of patient renal function, using the “Calvert formula”, an empirical carboplatin dosing formula described in A. H. Calvert et al., “Carboplatin dosage: prospective evaluation of a simple formula based on renal function,” J. Clin. Oncol., 1989, vol. 7, 1748-1756, the disclosure of which is incorporated herein by reference in its entirety. In particular, the carboplatin dose is calculated as:

Dose=target AUC×(GFR+25)

where the target AUC is expressed in mg/mL/min, GFR is expressed in mL/min, and the dose is given in mg. GFR can be determined by methods well known in the art, such as by measurement of creatinine clearance or by estimation from serum creatinine values; see, e.g., R. W. Jelliffe, “Creatinine clearance: bedside estimate,” Annals of Internal Medicine, 79, 4:604, 1973, the disclosure of which is incorporated herein by reference in its entirety. Target AUCs can be from 4 to 7 mg/mL/min, preferably from 5 to 6 mg/mL/min. The dose of carboplatin can be administered by intravenous infusion over 30 minutes, once every 3 weeks, but should be given after the paclitaxel infusion.

Compound 1 can be administered without regard to order relative to the paclitaxel and carboplatin. Compound 1 can be administered continuously (i.e., daily for the duration of the treatment), or more preferably, in an intermittent dosing regimen, such as 4/2 or 2/1, and paclitaxel and carboplatin can be administered once every 3 weeks as described above without regard to the Compound 1 dosing schedule. Preferably, Compound 1 is administered on a 4/2 dosing schedule, and the dosing regimens are synchronized so that the paclitaxel, followed by carboplatin, treatment occurs on days 1 and 22 of each Compound 1 4/2 treatment cycle. Alternatively, Compound 1 can be administered on a 2/1 dosing schedule, and the dosing regimens are synchronized so that the paclitaxel, followed by carboplatin, treatment occurs on day 1 of each Compound 1 treatment cycle. Compound 1, paclitaxel and carboplatin can be administered in a fed or fasted state. In a particularly preferred embodiment, Compound 1 is administered in an amount of 25 to 50, preferably 25, 37.5 or 50 mg daily, on a 4/2 dosing schedule, paclitaxel is administered in an amount of 135 to 175 mg/m² once every three weeks, and carboplatin is administered in an amount calculated from the Calvert formula as described above based on a target AUC of 4 to 7 mg/mL/min, once every three weeks following the paclitaxel dose. In another particularly preferred embodiment, Compound 1 is administered in an amount of 25 to 50, preferably 25, 37.5 or 50 mg daily, on a continuous dosing schedule, paclitaxel is administered in an amount of 135 to 175 mg/m² once every three weeks, and carboplatin is administered in an amount calculated from the Calvert formula as described above based on a target AUC of 4 to 7 mg/mL/min, once every three weeks following the paclitaxel dose.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a 4/2 dosing schedule, paclitaxel in an amount of 135 to 175 mg/m² once every three weeks, and carboplatin in an amount calculated from the Calvert formula as described above based on a target AUC of 4 to 7 mg/mL/min, once every three weeks following the paclitaxel dose.

In another particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a continuous dosing schedule, paclitaxel in an amount of 135 to 175 mg/m² once every three weeks, and carboplatin in an amount calculated from the Calvert formula as described above based on a target AUC of 4 to 7 mg/mL/min, once every three weeks following the paclitaxel dose.

In another particularly preferred aspect of this embodiment, the invention provides a method of ovarian cancer in a patient, such as a human, by administering to the patient Compound 1 in an amount of 25 to 50, preferably 25, 37.5 or 50 mg daily, on a 4/2 dosing schedule, paclitaxel in an amount of 135 to 175 mg/m² once every three weeks, and carboplatin in an amount calculated from the Calvert formula as described above based on a target AUC of 4 to 7 mg/mL/min, once every three weeks following the paclitaxel dose.

In another particularly preferred aspect of this embodiment, the invention provides a method of ovarian cancer in a patient, such as a human, by administering to the patient Compound 1 in an amount of 25 to 50, preferably 25, 37.5 or 50 mg daily, on a continuous dosing schedule, paclitaxel in an amount of 135 to 175 mg/m² once every three weeks, and carboplatin in an amount calculated from the Calvert formula as described above based on a target AUC of 4 to 7 mg/mL/min, once every three weeks following the paclitaxel dose.

(18) Gemcitabine-Cisplatin: In another embodiment, the invention provides combination therapies of Compound 1, gemcitabine, and cisplatin. Gemcitabine is a compound available from Eli Lilly and Company as a lyophilate for injection in single-use vials containing 200 mg or 1 g (free base equivalent) gemcitabine HCl, under the tradename Gemzar™. Cisplatin is a compound available from Bristol Meyers-Squib in multi-dose vials containing 50 or 100 mg cisplatin, under the tradename Platinol™-AQ (cisplatin injection). Gemcitabine, diluted for infusion as directed by the manufacturer can be administered in a dosage of from 750 to 1250 mg/m², preferably 750, 1000 or 1250 mg/m², as a 30-minute bolus infusion once weekly for 2, 3 or 4 weeks, followed by a one-week rest period. Cisplatin can be administered in a dosage of from 50 to 100 mg/m² as a 1 to 4 hour intravenous infusion, once every 3 or 4 weeks. Various gemcitabine-cisplatin combination regimens are known, and one skilled in the art can choose an appropriate dose and schedule depending upon individual patient and disease factors. Suitable dosing regimens are described, for example, in H. S. Parra et al., “Three-wee versus four-week schedule of cisplatin and gemcitabine: results of a randomized phase II study,” Annals of Oncology 13: 1080-1086, 2002; D. Castellano et al., “A phase 11 study of a novel gemcitabine plus cisplatin regimen administered every three weeks for advanced non-small-cell lung cancer,” Annals of Oncology 9: 457-459, 1998; and J. R. Kroep et al., “Gemcitabine-cisplatin: a schedule finding study,” Annals of Oncology 10:1503-1510, 1999, the disclosures of which are incorporated herein by reference in their entireties. Compound 1 can be administered in a dosage of from 25 to 75 mg once daily, preferably 25, 37.5, 50, 62.5 or 75 mg once daily, preferably orally. Compound 1 can be administered continuously (i.e., daily for the duration of the treatment), or more preferably, in an intermittent dosing regimen, such as 4/2, 4/1, 3/1 or 2/1. Gemcitabine and cisplatin can be administered as described above without regard to the Compound 1 dosing schedule. More preferably, the Compound 1, gemcitabine and cisplatin dosing schedules are chosen to provide as much synchronization of treatment cycles as possible. Compound 1, paclitaxel and carboplatin can be administered in a fed or fasted state.

In a particularly preferred embodiment, Compound 1 is administered in an amount of 25 to 50, preferably 25, 37.5 or 50 mg daily, on a 4/2 dosing schedule, and gemcitabine and cisplatin are as described above.

In another particularly preferred embodiment, Compound 1 is administered in an amount of 25 to 50, preferably 25, 37.5 or 50 mg daily, on a continuous dosing schedule, and gemcitabine and cisplatin are as described above.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a 4/2 dosing schedule; gemcitabine in an amount of 750 to 1250 mg/m² once weekly for 2, 3 or 4 weeks followed by a one-week rest period; and cisplatin in an amount of 50 to 100 mg/m² once every 3 or 4 weeks.

In another particularly preferred aspect of this embodiment, the invention provides a method of treating non-small cell lung cancer in a patient, such as a human, by administering to the patient Compound 1 in an amount of 25 to 50, preferably 25, 37.5 or 50 mg daily, on a continuous dosing schedule; gemcitabine in an amount of 750 to 1250 mg/m² once weekly for 2, 3 or 4 weeks followed by a one-week rest period; and cisplatin in an amount of 50 to 100 mg/m² once every 3 or 4 weeks.

In another particularly preferred aspect of this embodiment, the invention provides a method of treating bladder cancer in a patient, such as a human, by administering to the patient Compound 1 in an amount of 25 to 50, preferably 25, 37.5 or 50 mg daily, on a 4/2 dosing schedule; gemcitabine in an amount of 750 to 1250 mg/m² once weekly for 2, 3 or 4 weeks followed by a one-week rest period; and cisplatin in an amount of 50 to 100 mg/m² once every 3 or 4 weeks.

In another particularly preferred aspect of this embodiment, the invention provides a method of treating bladder cancer in a patient, such as a human, by administering to the patient Compound 1 in an amount of 25 to 50, preferably 25, 37.5 or 50 mg daily, on a continuous dosing schedule; gemcitabine in an amount of 750 to 1250 mg/m² once weekly for 2, 3 or 4 weeks followed by a one-week rest period; and cisplatin in an amount of 50 to 100 mg/m² once every 3 or 4 weeks.

(19) Adriamycin™doxorubicin-cyclophosphamide: In another embodiment, the invention provides combination therapies of Compound 1, doxorubicin and cyclophosphamide. Doxorubicin is a compound available from Pharmacia & Upjohn Co. (Pfizer, Inc.) as a lyophilate for injection in single-use vials containing 10, 20 or 50 mg, and in a multiple-use vial containing 150 mg, of doxorubicin hydrochloride, under the tradename Adriamycin RDF™ (doxorubicin hydrochloride for injection). Doxorubicin is also available from Pharmacia & Upjohn Co. (Pfizer, Inc.) as a sterile, parenteral, isotonic solution for IV use in 5 mL (10 mg), 10 mL (20 mg), 25 mL (50 mg) and 37.5 mL (75 mg) single-dose vials and a 100 mL (200 mg) multidose vial, under the tradename Adriamycin PFS™ (doxorubicin hydrochloride for injection). Cyclophosphamide is a compound available from Bristol-Myers Squibb as a lyophilate for injection in various strengths (e.g., 100 mg, 200 mg, 500 mg, 1 g and 2 g) under the tradename Cytoxan™ (cyclophosphamide for injection), for injection or intravenous infusion. Cyclophosphamide is also available in 25 and 50 mg (anhydrous) tablets for oral use under the tradename Cytoxan™ (cyclophosphamide tablets). Preferably, the combination is used to treat a patient, preferably a human, suffering from cancer. Thus, in a particular aspect of this embodiment, the invention provides a method of treating cancer by administering to a patient Compound 1, doxorubicin and cyclophosphamide in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Doxorubicin can be administered in a dosage of from 40 to 75 mg/m², preferably 40, 60 or 75 mg/m², as a single intravenous injection, once every 3 or 4 weeks. Cyclophosphamide can be administered in a variety of dose amounts and schedules well known in the art, preferably in a dosage of from 400 to 800 mg/m², preferably 600 mg/m², as a single intravenous injection, once every 3 or 4 weeks. Compound 1, doxorubicin and cyclophosphamide can be administered without regard to order. Compound 1 can be administered continuously (i.e., daily for the duration of the treatment), or more preferably, in an intermittent dosing regimen, such as 4/2, 3/1 or 2/1. Doxorubicin can be administered once every 3 weeks or once every 4 weeks without regard to the Compound 1 or cyclophosphamide dosing schedule, but preferably on the same dosing schedule as cyclophosphamide. Cyclophosphamide can be administered once every 3 weeks or once every 4 weeks without regard to the Compound 1 or doxorubicin dosing schedule, but preferably on the same dosing schedule as doxorubicin. Preferably, doxorubicin is administered once every 3 weeks, cyclophosphamide is administered once every 3 weeks, and Compound 1 is administered on a 4/2 dosing schedule, with the dosing regimens synchronized so that the doxorubicin and cyclophosphamide treatments occur on days 1 and 22 of each Compound 1 4/2 treatment cycle. Alternatively, Compound 1 is administered on a 2/1 dosing schedule, and the dosing regimens are synchronized so that the doxorubicin and cyclophosphamide treatments occur on day 1 of each Compound 1 2/1 treatment cycle. Compound 1, doxorubicin and cyclophosphamide can be administered in a fed or fasted state.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating breast cancer in a patient, such as a human, by administering to the patient Compound 1 in an amount of 25 to 50, preferably 25, 37.5 or 50 mg daily, on a 4/2 dosing schedule, doxorubicin in an amount of 40 to 75 mg/m², preferably 40, 60 or 75 mg/m² once every 3 or 4 weeks, and cyclophosphamide in an amount of 400 to 800 mg/m², preferably 400, 600 or 800 mg/m² once every 3 or 4 weeks.

In another particularly preferred aspect of this embodiment, the invention provides a method of treating breast cancer in a patient, such as a human, by administering to the patient Compound 1 in an amount of 25 to 50, preferably 25, 37.5 or 50 mg daily, on a continuous dosing schedule, doxorubicin in an amount of 40 to 75 mg/m², preferably 40, 60 or 75 mg/m² once every 3 or 4 weeks, and cyclophosphamide in an amount of 400 to 800 mg/m^{2,} preferably 400, 600 or 800 mg/m² once every 3 or 4 weeks.

(20) 5-Fluorouracil-epirubicin-cyclophosphamide: In another embodiment, the invention provides combination therapies of Compound 1 and 5-fluorouracil (“5-FU”), epirubicin and cyclophosphamide. 5-Fluorouracil is available as a solution for injection in 500 mg vials (50 mg/mL, 10 mL) from Pfizer, Inc., under the tradename Adrucil™. Epirubicin is a compound available from Pharmacia & Upjohn Co. (Pfizer, Inc.) as a sterile solution for IV use in vials containing 50 or 200 mg epirubicin hydrochloride under the tradename Ellence™ (epirubicin hydrochloride injection). Cyclophosphamide is a compound available from Bristol-Myers Squibb as a lyophilate for injection in various strengths (e.g., 100 mg, 200 mg, 500 mg, 1 g and 2 g) under the tradename Cytoxan™ (cyclophosphamide for injection), for injection or intravenous infusion. Cyclophosphamide is also available in 25 and 50 mg (anhydrous) tablets for oral use under the tradename Cytoxan™ (cyclophosphamide tablets). Preferably, the combination is used to treat a patient, preferably a human, suffering from cancer. Thus, in a particular aspect of this embodiment, the invention provides a method of treating cancer by administering to a patient Compound 1, 5-fluorouracil, epirubicin and cyclophosphamide in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Compound 1 can be administered continuously (i.e., daily for the duration of the treatment), or more preferably, in an intermittent dosing regimen, in a fed or fasted state. 5-FU can be administered according to dosing schedules well known in the art. For example, 5-FU can be administered on days 1 and 2 of each two week cycle, as an intravenous bolus of 400 mg/m² 5-FU and 600 mg/m² of 5-FU in a 22-hour infusion. Epirubicin can be administered in a dosage of from 60 to 120 mg/m^{2,} preferably 60, 75, 90, 100 or 120 mg/m², preferably by injection into a freely flowing intravenous infusion (0.9% sodium chloride or 5% glucose solution) over a period of 3 to 5 minutes, once every 3 or 4 weeks. Optionally, the total dose of epirubicin in each 3 or 4 week cycle can be divided equally and given on days 1 and 8 of each cycle. Cyclophosphamide can be administered in a variety of dose amounts and schedules well known in the art, preferably in a dosage of from 400 to 800 mg/m², preferably 600 mg/m², as a single intravenous injection, once every 3 or 4 weeks. Compound 1, 5-FU, epirubicin and cyclophosphamide can be administered without regard to order. Preferably, the treatment schedules of the various agents are synchronized so that treatment days for 5-FU, epirubicin and cyclophosphamide coincide as much as possible.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating breast cancer in a patient, such as a human, by administering to the patient Compound 1 in an amount of 25 to 50, preferably 25, 37.5 or 50 mg daily, on a 4/2 or 2/2 dosing schedule, and 5-fluorouracil, epirubicin and cyclophosphamide on dosing schedules as described above.

In another particularly preferred aspect of this embodiment, the invention provides a method of treating breast cancer in a patient, such as a human, by administering to the patient Compound 1 in an amount of 25 to 50, preferably 25, 37.5 or 50 mg daily, on a continuous dosing schedule, and 5-fluorouracil, epirubicin and cyclophosphamide on dosing schedules as described above.

(21) Herceptin™/trastuzumab-paclitaxel: In another embodiment, the invention provides combination therapies of Compound 1, trastuzumab and paclitaxel. Trastuzumab is a monoclonal antibody available from Genentech as a lyophilate for injection in single-use vials containing 440 mg trastuzumab, under the tradename Herceptin™. Paclitaxel is a compound available from Mead Johnson as a non-aqueous solution for dilution in 30, 100 and 300 mL multi-dose vials under the tradename Taxol™. Preferably, the combination is used to treat a patient, preferably a human, suffering from cancer. Thus, in a particular aspect of this embodiment, the invention provides a method of treating cancer by administering to a patient Compound 1, trastuzumab and paclitaxel in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Trastuzumab, diluted for infusion as directed by the manufacturer can be administered in an initial loading dose of 4 mg/kg as a 90-minute infusion, followed by once-weekly maintenance doses of 2 mg/kg as a 30-minute infusion for the duration of the treatment. Paclitaxel, diluted for infusion as directed by the manufacturer, can be administered in a dosage of from 135 to 175 mg/m² as an infusion over 3 hours, once every 3 weeks. Compound 1, trastuzumab and paclitaxel can be administered without regard to order. Compound 1 can be administered continuously (i.e., daily for the duration of the treatment), or more preferably, in an intermittent dosing regimen, such as 4/2 or 2/1, paclitaxel can be administered once every 3 weeks as described above without regard to the Compound 1 and trastuzumab dosing schedules, and trastuzumab can be administered once per week without regard to the Compound 1 and paclitaxel dosing schedules. Preferably, Compound 1 is administered on a 4/2 dosing schedule, and the dosing regimens are synchronized so that the paclitaxel treatment occurs on days 1 and 22 of each Compound 1 4/2 treatment cycle, with trastuzumab once weekly. Alternatively, Compound 1 can be administered on a 2/1 dosing schedule, and the dosing regimens are synchronized so that the paclitaxel treatment occurs on day 1 of each Compound 1 treatment cycle, with trastuzumab once weekly. Compound 1, trastuzumab and paclitaxel can be administered in a fed or fasted state. In a particularly preferred embodiment, Compound 1 is administered in an amount of 25 to 50, preferably 25, 37.5 or 50 mg daily, on a 4/2 dosing schedule, trastuzumab is administered once weekly, and paclitaxel is administered once every 3 weeks, as described above. In another particularly preferred embodiment, Compound 1 is administered in an amount of 25 to 50, preferably 25, 37.5 or 50 mg daily, on a continuous dosing schedule, trastuzumab is administered once weekly, and paclitaxel is administered once every 3 weeks, as described above.

In a particular aspect of this embodiment, the cancer is breast cancer, particularly HER2 positive breast cancer. As used herein, the term “HER2 positive” means characterized by HER2 protein overexpression, and such overexpression can be determined by methods well known in the art, such as by immunohistochemistry (IHC) or fluorescence in situ hybridization (FISH). A HER2 IHC assay is available commercially from DakoCytomation, Carpinteria, Calif., USA, under the tradename HercepTest™. A HER2 FISH assay is available commercially from Vysis, Inc., Downers Grove, Ill., USA, under the tradename PathVysion™. HER2 assays are described in the literature in, for example, M. F. Press et al., “Her-2/neu expression in noe-negative breast cancer: direct tissue quantitation by computerized image analysis and association of overexpression with increased risk of recurrent disease,” Cancer Res. 1993, 53, 4960-4970, the disclosure of which is incorporated herein by reference in its entirety.

In a particularly preferred aspect of this embodiment, the invention provides a method of treating HER2 positive breast cancer in a patient, such as a human, by administering to the patient Compound 1 in an amount of 25 to 50, preferably 25, 37.5 or 50 mg daily, on a 4/2 dosing schedule, paclitaxel in an amount of 135 to 175 mg/m² by infusion once every 3 weeks, and trastuzumab in an initial loading dose of 4 mg/kg followed by once-weekly doses of 2 mg/kg by infusion.

In another particularly preferred aspect of this embodiment, the invention provides a method of treating HER2 positive breast cancer in a patient, such as a human, by administering to the patient Compound 1 in an amount of 25 to 50, preferably 25, 37.5 or 50 mg daily, on a continuous dosing schedule, paclitaxel in an amount of 135 to 175 mg/m² by infusion once every 3 weeks, and trastuzumab in an initial loading dose of 4 mg/kg followed by once-weekly doses of 2 mg/kg by infusion.

(22) IFL: In another embodiment, the invention provides combination therapies of Compound 1 and IFL, a combination of irinotecan, 5-fluorouracil (“5-FU”) and leucovorin. Irinotecan, also known as CPT-11, is available from Pfizer, Inc. as a solution for dilution and injection in 2 and 5 mL vials (40 and 100 mg irinotecan hydrochloride, respectively) under the tradename Camptosar™ (irinotecan hydrochloride injection). 5-Fluorouracil is available as a solution for injection in 500 mg vials (50 mg/mL, 10 mL) from Pfizer, Inc., under the tradename Adrucil™. Leucovorin, also known as LV, calcium leucovorin, folinic acid, calcium folinate or citrovorum factor, is available from several sources, including Wyeth Pharmaceuticals (Lederle Leucovorin™ Calcium). Preferably, the combination is used to treat a patient, preferably a human, suffering from cancer. Thus, in a particular aspect of this embodiment, the invention provides a method of treating cancer by administering to a patient Compound 1 and IFL in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Compound 1 can be administered continuously (i.e., daily for the duration of the treatment), or more preferably, in an intermittent dosing regimen, in a fed or fasted state. Irinotecan, 5-FU and leucovorin can be administered according to the standard IFL dosing schedule well known in the art”. In particular, IFL can be administered in 6-week cycles (4/2), as follows. Once per week for 4 weeks, 100-125 mg/m² irinotecan is administered in a 90-minute infusion, followed by 20 mg/m² leucovorin and then 400-500 mg/m² 5-FU. The 4 weeks of IFL treatment are followed by a 2-week IFL rest period. In a preferred embodiment, Compound 1 is administered in an amount of 25 to 50, preferably 25, 37.5 or 50 mg daily, on a 4/2 dosing schedule (daily), and IFL is administered on a 4/2 dosing schedule (weekly) as described herein. Preferably, the Compound 1 and IFL cycles are synchronized so that the treatment periods of Compound 1 and IFL coincide, and the rest periods of Compound 1 and IFL coincide. In another preferred embodiment, Compound 1 is administered in an amount of 25 to 50, preferably 25, 37.5 or 50 mg daily, on a continuous dosing schedule (daily), and IFL is administered on a 4/2 dosing schedule (weekly) as described herein.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a 4/2 dosing schedule, and IFL on a standard IFL dosing regimen as described above.

In another particularly preferred aspect of this embodiment, the invention provides a method of treating colorectal cancer in a patient, such as a human, by administering to the patient Compound 1 in an amount of 25 to 50, preferably 25, 37.5 or 50 mg daily, on a continuous dosing schedule, and IFL on a standard IFL dosing regimen as described above.

(23) MEK Inhibitors: In another embodiment, the invention provides combination therapies of Compound 1 and a MEK inhibitor. Preferred MEK inhibitors include those disclosed in PCT Publication No. WO 02/06213. A particularly preferred MEK inhibitor is N—[(R)-2,3-dihydroxy-propoxy]-3,4-difluoro-2-(2-fluoro-4-iodo-phenylamino)-benzamide, also known as PD325901, a MEK inhibitor currently in clinical development by Pfizer. PD325901 can be prepared as described in WO 02/02613. Preferably, the combination is used to treat a patient, preferably a human, suffering from cancer. Thus, in a particular aspect of this embodiment, the invention provides a method of treating cancer by administering to a patient Compound 1 and a MEK inhibitor, preferably PD325901, in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Compound 1 can be administered continuously (i.e., daily for the duration of the treatment), or more preferably, in an intermittent dosing regimen, in a fed or fasted state. Preferred dosing regimens of PD325901 are described in U.S. Provisional Application No. 60/648,972, filed Jan. 31, 2005. For example, PD325901 can be administered in a dosage of from 10 to 30 mg orally once daily or twice daily, preferably orally. PD325901 can be administered continuously (i.e., once or twice daily for the duration of the treatment), or in an intermittent dosing regimen, such as a 4/2, 4/1 or 3/1 dosing regimen. In a preferred embodiment, Compound 1 is administered in an amount of 25 to 50, preferably 25, 37.5 or 50 mg daily, on a 4/2 dosing schedule (daily), and PD325901 is administered in an amount of 10 or 15 or 20 or 25 or 30 mg twice daily on a continuous dosing schedule. Preferably, the Compound 1 and MEK inhibitor cycles are synchronized so that the treatment periods of Compound 1 and the MEK inhibitor, and the rest periods of Compound 1 and the MEK inhibitor coincide as much as possible. In another preferred embodiment, Compound 1 is administered in an amount of 25 to 50, preferably 25, 37.5 or 50 mg daily, on a continuous dosing schedule (daily), and PD325901 is administered in an amount of 10 or 15 or 20 or 25 or 30 mg twice daily on a continuous dosing schedule.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating melanoma in a patient, such as a human, by administering to the patient Compound 1 in an amount of 25 to 50, preferably 25, 37.5 or 50 mg daily, on a 4/2 dosing schedule, and PD325901 in an amount of 10 or 15 or 20 or 25 or 30 mg twice daily on a continuous dosing schedule.

In another particularly preferred aspect of this embodiment, the invention provides a method of treating melanoma in a patient, such as a human, by administering to the patient Compound 1 in an amount of 25 to 50, preferably 25, 37.5 or 50 mg daily, on a continuous dosing schedule, and PD325901 in an amount of 10 or 15 or 20 or 25 or 30 mg twice daily on a continuous dosing schedule.

(24) Taxotere™/docetaxel-prednisone: In another embodiment, the invention provides combination therapies of Compound 1, docetaxel, an antineoplastic agent available from Aventis Pharmaceuticals as an injection concentrate in single-use vials containing 20 mg (0.5 mL) or 80 mg (2 mL) docetaxel (anhydrous), under the tradename Taxotere™; and a glucocorticosteroid such as prednisone or prednisolone. Preferably, the combination is used to treat a patient, preferably a human, suffering from cancer, particularly prostate cancer. Thus, in a particular aspect of this embodiment, the invention provides a method of treating prostate cancer by administering to a patient Compound 1, docetaxel and prednisone or prednisolone in amounts that in combination are therapeutically effective. In this embodiment, Compound 1 can be administered in a dosage of from 12.5 or 25 to 75 mg once daily, preferably 12.5, 25, 37.5, 50, 62.5 or 75 mg once daily, preferably orally. Docetaxel, diluted for infusion as directed by the manufacturer can be administered in a dosage of from 60 to 100 mg/m², preferably 60, 75 or 100 mg/m², as a 60-minute intravenous infusion once every three weeks. Prednisone can be administered in an amount of 5 mg twice daily, on a continuous dosing schedule. Compound 1, docetaxel and prednisone can be administered at the same time, or sequentially, without regard to order. Compound 1 can be administered continuously (i.e., daily for the duration of the treatment), or more preferably, in an intermittent dosing regimen. Docetaxel can be administered once every three weeks without regard to the Compound 1 dosing schedule. In a particularly preferred embodiment, Compound 1 is administered in an amount of 25 to 50, preferably 25, 37.5 or 50 mg daily, on a 4/2 or 2/1 dosing schedule, docetaxel is administered in an infusion of 60, 75 or 100 mg/m² once every three weeks, and prednisone is administered in an amount of 5 mg twice daily on a continuous dosing schedule. In another particularly preferred embodiment, Compound 1 is administered in an amount of 25 to 50, preferably 25, 37.5 or 50 mg daily, on a continuous dosing schedule, docetaxel is administered in an infusion of 60, 75 or 100 mg/m² once every three weeks, and prednisone is administered in an amount of 5 mg twice daily on a continuous dosing schedule.

In a particularly preferred aspect of this embodiment, the invention provides a method of treating prostate cancer in a patient, such as a human, by administering to the patient Compound 1 in an amount of 25 to 50, preferably 25, 37.5 or 50 mg daily, on a 4/2 or 2/1 dosing schedule, docetaxel in an infusion of 75 mg/m² once every three weeks, and prednisone in an amount of 5 mg twice daily on a continuous dosing schedule.

In a particularly preferred aspect of this embodiment, the invention provides a method of treating prostate cancer in a patient, such as a human, by administering to the patient Compound 1 in an amount of 12.5 to 50, preferably 12.5, 25, 37.5 or 50 mg daily, on a 4/2 or 2/1 dosing schedule, docetaxel in an infusion of 60 mg/m² once every three weeks, and prednisone in an amount of 5 mg twice daily on a continuous dosing schedule.

In another particularly preferred aspect of this embodiment, the invention provides a method of treating prostate cancer in a patient, such as a human, by administering to the patient Compound 1 in an amount of 25 to 50, preferably 25, 37.5 or 50 mg daily, on a continuous dosing schedule, docetaxel in an infusion of 75 mg/m² once every three weeks, and prednisone in an amount of 5 mg twice daily on a continuous dosing schedule.

In another particularly preferred aspect of this embodiment, the invention provides a method of treating prostate cancer in a patient, such as a human, by administering to the patient Compound 1 in an amount of 12.5 to 50, preferably 12.5, 25, 37.5 or 50 mg daily, on a continuous dosing schedule, docetaxel in an infusion of 60 mg/m² once every three weeks, and prednisone in an amount of 5 mg twice daily on a continuous dosing schedule.

(25) Anti-androgens: In another embodiment, the invention provides combination therapies of Compound 1 and an anti-androgen. Suitable anti-androgens include bicalutamide, a compound available as a 150 mg tablet from Aztra-Zeneca under the tradename Casodex™; flutamide, a compound available as a 125 mg capsule from Schering under the tradename Eulexin™; and nilutamide, a compound available as a 150 mg tablet from Aventis under the tradename Nilandron™. Preferably, the combination is used to treat a patient, preferably a human, suffering from cancer, particularly prostate cancer. Thus, in a particular aspect of this embodiment, the invention provides a method of treating prostate cancer by administering to a patient Compound 1 and an anti-androgen, such as bicalutamide, flutamide or nilutamide, in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Bicalutamide can be administered once daily in an amount of 150 mg on a continuous dosing schedule. Flutamide can be administered in an amount of 250 mg three times daily on a continuous dosing schedule. Nilutamide can be administered once daily in an amount of from 150 to 300 mg on a continuous dosing schedule. Compound 1 and the anti-androgen can be administered at the same time, or sequentially, without regard to order. Compound 1 can be administered continuously (i.e., daily for the duration of the treatment), or more preferably, in an intermittent dosing regimen

In a particularly preferred aspect of this embodiment, the invention provides a method of treating prostate cancer in a patient, such as a human, by administering to the patient Compound 1 in an amount of 25 to 50, preferably 25, 37.5 or 50 mg daily, on a 4/2 dosing schedule, and an anti-androgen on a continuous dosing schedule.

In another particularly preferred aspect of this embodiment, the invention provides a method of treating prostate cancer in a patient, such as a human, by administering to the patient Compound 1 in an amount of 25 to 50, preferably 25, 37.5 or 50 mg daily, on a continuous dosing schedule, and an anti-androgen on a continuous dosing schedule.

(26) LHRH agonists or antagonists: In another embodiment, the invention provides combination therapies of Compound 1 and a luteinizing hormone-releasing hormone (LHRH) agonist or antagonist. Suitable LHRH agonists include leuprolide, a compound available as an acetate salt in 7.5, 22.5 and 30 mg dosages from TAP Pharmaceuticals under the tradename LupronDepot™; and goserelin, a compound available as an acetate salt in a 10.8 mg depot from AstraZeneca under the tradename Zoladex™. Suitable LHRH antagonists include abarelix, a compound available from Praecix under the tradename Plenaxis™. Preferably, the combination is used to treat a patient, preferably a human, suffering from cancer, particularly prostate cancer. Thus, in a particular aspect of this embodiment, the invention provides a method of treating prostate cancer by administering to a patient Compound 1 and an LHRH agonist or antagonist, such as leuprolide, goserilin or abarelix, in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Leuprolide can be administered once monthly in an amount of 7.5 mg (LupronDepot™ 7.5 mg), or once every 3 months in an amount of 22.5 mg (LupronDepot™ 22.5 mg) or once every 4 months in an amount of 30 mg (LupronDepot™ 30 mg). Goserelin can be administered in an amount of 10.8 mg once every 3 months. Abarelix can be administered once every 4 weeks in an amount of 100 mg. Compound 1 and the LHRH agonist or antagonist can be administered at the same time, or sequentially, without regard to order. Compound 1 can be administered continuously (i.e., daily for the duration of the treatment), or more preferably, in an intermittent dosing regimen In a particularly preferred aspect of this embodiment, the invention provides a method of treating prostate cancer in a patient, such as a human, by administering to the patient Compound 1 in an amount of 25 to 50, preferably 25, 37.5 or 50 mg daily, on a 4/2 dosing schedule, and an LHRH agonist or antagonist.

In another particularly preferred aspect of this embodiment, the invention provides a method of treating prostate cancer in a patient, such as a human, by administering to the patient Compound 1 in an amount of 25 to 50, preferably 25, 37.5 or 50 mg daily, on a continuous dosing schedule, and an LHRH agonist or antagonist.

(27) Nexavar™/sorafenib: In another embodiment, the invention provides combination therapies of Compound 1 and sorafenib, a multi-kinase inhibitor available from Onyx Pharmaceuticals as a 200 mg tablet (free base equivalent) of the tosylate salt under the tradename Nexavar™. Preferably, the combination is used to treat a patient, preferably a human, suffering from cancer. Thus, in a particular aspect of this embodiment, the invention provides a method of treating cancer by administering to a patient Compound 1 and sorafenib in amounts that in combination are therapeutically effective. In this embodiment, Compound 1 can be administered in a dosage of from 25 to 75 mg once daily, preferably 25, 37.5, 50, 62.5 or 75 mg once daily, preferably orally. Compound 1 can be administered continuously (i.e., daily for the duration of the treatment) or in an intermittent dosing regimen, in a fed or fasted state. Sorafenib can be administered in an amount of from 200 mg to 400 mg, twice daily or once daily or once every two days, in a fasted state. In a preferred embodiment, Compound 1 is administered in an amount of 25 to 50, preferably 25, 37.5 or 50 mg daily, on a 4/2 or 2/1 dosing schedule, and sorafenib is administered on a continuous dosing schedule, preferably 400 mg twice daily. In another preferred embodiment, Compound 1 is administered in an amount of 25 to 50, preferably 25, 37.5 or 50 mg daily, on a continuous dosing schedule (daily), and sorafenib is administered on a continuous dosing schedule, preferably 400 mg twice daily.

In particular aspects of this embodiment, the cancer is lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. More preferably, the cancer is gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, pancreatic cancer, or a combination thereof.

In a particularly preferred aspect of this embodiment, the invention provides a method of treating renal cell carcinoma in a patient, such as a human, by administering to the patient Compound 1 in an amount of 25 to 50, preferably 25, 37.5 or 50 mg daily, on a 4/2 dosing schedule, and sorafenib in an amount of 200 mg or 400 mg, twice daily or once daily or once every two days.

In another particularly preferred aspect of this embodiment, the invention provides a method of treating renal cell carcinoma in a patient, such as a human, by administering to the patient Compound 1 in an amount of 25 to 50, preferably 25, 37.5 or 50 mg daily, on a continuous dosing schedule, and sorafenib in an amount of 200 mg or 400 mg, twice daily or once daily or once every two days.

For administration to the eye, Compound 1 is delivered in a pharmaceutically acceptable ophthalmic vehicle such that the compound is maintained in contact with the ocular surface for a sufficient time period to allow the compound to penetrate the cornea and/or sclera and internal regions of the eye, including, for example, the anterior chamber, posterior chamber, vitreous body, aqueous humor, vitreous humor, cornea, iris/cilary, lens, choroid/retina and sclera. The pharmaceutically acceptable ophthalmic vehicle may be an ointment, vegetable oil, or an encapsulating material. Compound 1 can alternatively be injected directly into the vitreous humor or aqueous humor.

Compound 1 may be administered to the eye by any of a variety of well known methods, such as by subtenon and/or subconjunctival injections. As is well known in the ophthalmic art, the macula is formed primarily of retinal cones and is the region of maximum visual acuity in the retina. A Tenon's capsule or Tenon's membrane is disposed on the sclera. A conjunctiva covers a short area of the globe of the eye posterior to the limbus (the bulbar conjunctiva) and folds up (the upper cul-de-sac) or down (the lower cul-de-sac) to cover the inner areas of the upper eyelid and lower eyelid, respectively. The conjunctiva is disposed on top of Tenon's capsule.

The sclera and Tenon's capsule define the exterior surface of the globe of the eye. For treatment of ARMD, CNV, retinopathies, retinitis, uveitis, cystoid macular edema (CME), and other diseases or conditions of the posterior segment of the eye, it is preferable to dispose a depot of a specific quantity of an ophthalmically acceptable pharmaceutically active agent directly on the outer surface of the sclera and below Tenon's capsule. In addition, in cases of ARMD and CME it is most preferable to dispose the depot directly on the outer surface of the sclera, below Tenon's capsule (sub-Tenon), and generally above the macula.

In addition to the formulations described above, Compound 1 may also be formulated as a depot preparation. Such long-acting formulations may be administered by implantation (for example, subcutaneously or intramuscularly) intramuscular injection or by the above mentioned subtenon or intravitreal injection.

Compound I can be prepared for topical administration in saline (combined with any of the preservatives and antimicrobial agents commonly used in ocular preparations), and administered in eye drop form. Suitable compositions can also be administered directly to the cornea.

A suitable ophthalmic composition can be prepared with a muco-adhesive polymer which binds to cornea. Thus, for example, Compound I can be formulated with suitable polymeric or hydrophobic materials (for example, as an emulsion in an acceptable oil) or ion-exchange resins, or as sparingly soluble derivatives, for example, as a sparingly soluble salt.

All patents, patent applications and publications referred to are incorporated herein by reference in their entireties.

We claim:

1. A method of treating a cancer in a patient comprising administering to the patient sunitinib malate in an amount of 25 to 50 mg free base equivalent daily and at least one additional therapeutic agent.

2. The method of claim 1, wherein the cancer is non-small cell lung cancer and the at least one additional therapeutic agent is:
(a) gefitinib daily in an amount of 250 mg; or
(b) erlotinib daily in an amount of 150 mg; or
(c) docetaxel in an infusion of 60 to 100 mg/m² once every three weeks; or
(d) gemcitabine in an infusion of 750 to 1250 mg/m² once weekly on a 4/1, 3/1 or 2/1 weekly dosing schedule; or
(e) pemetrexed in an amount of 250 to 500 mg/m² once every three weeks; or
(f) bevacizumab in an amount of 3 to 10 mg/kg once every two weeks; or
(g) paclitaxel in an amount of 135 to 175 mg/m² once every 3 weeks, and carboplatin in an amount sufficient to achieve a target AUC concentration of 4 to 7 mg/mL/min once every three weeks following the paclitaxel dose; or
(h) gemcitabine in an amount of 750 to 1250 mg/m² once weekly for 2, 3 or 4 weeks followed by a one-week rest period; and cisplatin in an amount of 50 to 100 mg/m² once every 3 or 4 weeks.

3. The method of claim 1, wherein the cancer is colorectal cancer and the at least one additional therapeutic agent is
(a) cetuximab in an initial infusion of 400 mg/m² followed by weekly infusions of 250 mg/m²; or
(b) capecitabine in an amount of 825 to 1250 mg/m² twice daily on a 2/1 dosing schedule; or
(c) oxaliplatin, 5-fluorouracil and leucovorin on a FOLFOX4 dosing schedule; or
(d) irinotecan, 5-fluorouracil and leucovorin on a FOLFIRI dosing schedule; or
(e) bevacizumab in an amount of 3 to 10 mg/kg once every two weeks; or
(f) irinotecan, 5-fluorouracil and leucovorin in an IFL dosing schedule; or
(g) a MEK inhibitor.

4. The method of claim 1, wherein the cancer is breast cancer and the at least one additional therapeutic agent is:
(a) docetaxel in an infusion of 60 to 100 mg/m² once every three weeks; or
(b) capecitabine in an amount of 825 to 1250 mg/m² twice daily on a 2/1 dosing schedule; or
(c) exemestane in an amount of 25 mg once daily; or
(d) trastuzumab on a once weekly dosing schedule; or
(e) anastrozole in an amount of 1 mg once daily; or
(e) letrozole in an amount of 2.5 mg once daily; or
(g) bevacizumab in an amount of 3 to 10 mg/kg once every two weeks; or
(h) doxorubicin in an amount of 40 to 75 mg/m² once every 3 or 4 weeks; and cyclophosphamide in an amount of 400 to 800 mg/m² once every 3 or 4 weeks; or
(g) 5-fluorouracil on an intermittent dosing schedule; epirubicin in an amount of 60 to 120 mg/m² once every 3 or 4 weeks; and cyclophosphamide in an amount of 400 to 800 mg/m² once every 3 or 4 weeks; or
(h) paclitaxel in an amount of 135 to 175 mg/m² once every 3 weeks; and trastuzumab in an amount 2 mg/kg once weekly.

5. The method of claim 1, wherein the cancer is prostate cancer and the at least one additional therapeutic agent is:
(a) docetaxel in an infusion of 60 to 100 mg/m² once every three weeks; or
(b) docetaxel in an amount of 75 mg/m² once every three weeks; and prednisone in an amount of 5 mg twice daily on a continuous dosing schedule; or
(c) an anti-androgen on a continuous dosing schedule; or
(d) an LHRH agonist or antagonist; or
(e) bevacizumab in an amount of 3 to 10 mg/kg once every two weeks.

6. The method of claim 1, wherein the cancer is renal cell carcinoma and the at least one additional therapeutic agent is:
(a) sunitinib malate in an amount of from 25 to 75 mg free base equivalent daily, and gefitinib daily in an amount of 250 mg; or
(b) sunitinib malate in an amount of from 25 to 50 mg free base equivalent daily, and erlotinib daily in an amount of 150 mg; or
(c) sunitinib malate in an amount of from 25 to 50 mg free base equivalent daily, and bevacizumab in an amount of 3 to 10 mg/kg once every two weeks.

7. The method of claim 1, wherein the cancer is pancreatic cancer and the at least one additional therapeutic agent is: (a) erlotinib daily in an amount of 150 mg; or (b) gemcitabine in an infusion of 750 to 1250 mg/m² once weekly on a 4/1, 3/1 or 2/1 weekly dosing schedule.

8. The method of claim 1, wherein the cancer is bladder cancer and the at least one additional therapeutic agent is: (a) gemcitabine in an infusion of 750 to 1250 mg/m² once weekly on a 4/1, 3/1 or 2/1 weekly dosing schedule; or (b) gemcitabine in an amount of 750 to 1250 mg/m² once weekly for 2, 3 or 4 weeks followed by a one-week rest period; and cisplatin in an amount of 50 to 100 mg/m² once every 3 or 4 weeks.

9. The method of claim 1, wherein the cancer is gastrointestinal stromal tumor and the at least one additional therapeutic agent is imatinib once daily in an amount of 400 to 600 mg on a continuous dosing schedule.

10. The method of claim 1, wherein the cancer is melanoma and the at least one additional therapeutic agent is: (a) temozolomide once daily in an amount of 150 to 200 mg/m² on the first 5 days of a 4-week dosing schedule; or (b) dacarbazine in an amount of 2 to 4.5 mg/kg/day on the first 10 days only of a 4-week treatment cycle or in an amount of 250 mg/m²/day on the first 5 days of a 3-week treatment cycle.

11. The method of claim 1, wherein the cancer is a sarcoma and the at least one additional therapeutic agent is: (a) doxorubicin in an amount of 40 to 75 mg/m² once every 3 or 4 weeks; or (b) epirubicin in an amount of 60 to 120 mg/m² once every 3 or 4 weeks.

12. The method of claim 1, wherein the cancer is ovarian cancer and the at least one additional therapeutic agent is paclitaxel in an amount of 135 to 175 mg/m² once every 3 weeks, and carboplatin in an amount sufficient to achieve a target AUC concentration of 4 to 7 mg/mL/min once every three weeks following the paclitaxel dose.
